SEQUENCE LISTING (22 sequences)

SEQ ID NO: 1 - protein, 163 aa, Staphylococcus aureus
MLKRSLLFLTVLLLLFSFSSITNEVSASSSFDKGKYKKGDDASYFEPTGPYLMVNVTGVDGKGNELLSPHYVEFPIKPGTTLTKEKIEYYVEWALDATAYKEFRVVELDPSAKIEVTYYDKNKKKEETKSFPITEKGFVVPDLSEHIKNPGFNLITKVVIEKK

SEQ ID NO: 2 - protein, 136 aa, Staphylococcus aureus
SSSFDKGKYKKGDDASYFEPTGPYLMVNVTGVDGKGNELLSPHYVEFPIKPGTTLTKEKIEYYVEWALDATAYKEFRVVELDPSAKIEVTYYDKNKKKEETKSFPITEKGFVVPDLSEHIKNPGFNLITKVVIEKK

SEQ ID NO: 3 - protein, 136 aa, Staphylococcus aureus
SSSFDKGKYKKGDDASYFEPTGPYLMVNVTGVDSKGNELLSPHYVEFPIKPGTTLTKEKIEYYVEWALDATAYKEFRVVELDPSAKIEVTYYDKNKKKEETKSFPITEKGFVVPDLSEHIKNPGFNLITKVVIEKK

SEQ ID NO: 4 - DNA, 42 bp, Artificial Sequence
  Description of Artificial Sequence Primer
aaaaagggcgatgacgcgaagtattttgaaccaacaggcccg

SEQ ID NO: 5 - DNA, 42 bp, Artificial Sequence
  Description of Artificial Sequence Primer
aaaaagggcgatgacgcgagtaagtttgaaccaacaggcccg

SEQ ID NO: 6 - DNA, 42 bp, Artificial Sequence
  Description of Artificial Sequence Primer
aaaaagggcgatgacgcgagttataaggaaccaacaggcccg

SEQ ID NO: 7 - DNA, 42 bp, Artificial Sequence
  Description of Artificial Sequence Primer
aaaaagggcgatgacgcgagttattttgaaccaacaggcccg

SEQ ID NO: 8 - DNA, 66 bp, Artificial Sequence
  Description of Artificial Sequence Primer
cggaattcttatttcttttctataacaacctttgtaattaagttgaacttagggtttttaatatgc

SEQ ID NO: 9 - DNA, 66 bp, Artificial Sequence
  Description of Artificial Sequence Primer
cggaattcttatttcttttctataacaacctttgtaattaagttctttccagggtttttaatatgc

SEQ ID NO: 10 - DNA, 66 bp, Artificial Sequence
  Description of Artificial Sequence Primer
cggaattcttatttcttttctataacaacctttgtaattaacttgaatccagggtttttaatatgc

SEQ ID NO: 11 - DNA, 66 bp, Artificial Sequence
  Description of Artificial Sequence Primer
cggaattcttatttcttttctataacaacctttgtctttaagttgaatccagggtttttaatatgc

SEQ ID NO: 12 - DNA, 66 bp, Artificial Sequence
  Description of Artificial Sequence Primer
cggaattcttatttcttttctataacaacctttgtaattaagttgaatccagggtttttaatatgc

SEQ ID NO: 13 - DNA, 69 bp, Artificial Sequence
  Description of Artificial Sequence Primer
ccaagcgctaagatcgaagtcacttattatgataataaagaagaaacgtctttccctataacagaaaaa

SEQ ID NO: 14 - DNA, 29 bp, Artificial Sequence
  Description of Artificial Sequence Primer
cggaattcttatttcttttctataacaac

SEQ ID NO: 15 - DNA, 1377 bp, Staphylococcus aureus
gtatacgcgctggaacattaatatatgtgtttgaaattatagatggttggtgtcgcatttattggaacaatcataatgagtggatatggcatgagagattgattgtgaaagaagtgttttaattcttaggttaaaatgttaaatatttgttaattatttttgaatgtaagtttagtttcttttaatattttattgatttttaatattttctcaatataaaatgaagttgttgatatttatcatcttaaataagggtgttagctataaaaagagataaataaaaacaaatatattatatttggaggaagcgccatgctcaaaagaagtttattatttttaactgttttattgttattattctcattttcttcaattactaatgaggtaagtgcatcaagttcattcgacaaaggaaaatataaaaagggcgatgacgcgagttattttgaaccaacaggcccgtatttgatggtaaatgtgactggagttgatggtaaaggaaatgaattgctatcccctcattatgtcgagtttcctattaaacctgggactacacttacaaaagaaaaaattgaatactatgtcgaatgggcattagatgcgacagcatataaagagtttagagtagttgaattagatccaagcgcaaagatcgaagtcacttattatgataagaataagaaaaaagaagaaacgaagtctttccctataacagaaaaaggttttgttgtcccagatttatcagagcatattaaaaaccctggattcaacttaattacaaaggttgttatagaaaagaaataaaacaaaatagttgtttattatagaaagtaatgtcttgattgaatatgtgtagtgaaattatctttcatcaaattctcattcatgcacgaatggttctgccccacctaatcagatattaggtgacttatggggagaaatcagttagaatgacatagtcatgtctatttaagcaggtgcgttacacacctgatgtctatttacatttaaagataaaatgtgctattattttactagaactttttaacatttctctcaagatttaaatgtagataacaggcaggtactacggtacttgcctgtttttttatgttatagctagccttcgggcagtttttgttatgatgcgttacacacgcatcaactattcacacctatctttgttcacctaagcatgtcactgggtgtttttttcttacgatagagagcatagttttcatactactccccgtagtatatatgactttagcattcccgtataacagtttacggggtgctttttatgttatacttacttttatatagtaggagtggactatatagctggtcagaggctgtatatctgactgttggtcc

SEQ ID NO: 16 - protein, 6 aa, Artificial Sequence
  Description of Artificial Sequence Synthetic peptide tag
HHHHHH

SEQ ID NO: 17 - protein, 8 aa, Artificial Sequence
  Description of Artificial Sequence Synthetic peptide tag
MKHHHHHH

SEQ ID NO: 18 - protein, 10 aa, Artificial Sequence
  Description of Artificial Sequence Synthetic peptide tag
MKHHAHHQHH

SEQ ID NO: 19 - protein, 14 aa, Artificial Sequence
  Description of Artificial Sequence Synthetic peptide tag
MKHQHQHQHQHQHQ

SEQ ID NO: 20 - protein, 10 aa, Artificial Sequence
  Description of Artificial Sequence Synthetic peptide tag
EQKLISEEDL

SEQ ID NO: 21 - protein, 8 aa, Artificial Sequence
  Description of Artificial Sequence Synthetic peptide tag
DYKDDDDK

SEQ ID NO: 22 - protein, 9 aa, Artificial Sequence
  Description of Artificial Sequence Synthetic peptide tag
YPYDVPDYA

(19) United States
(12) Patent Application Publication
(10) Pub. No.: US 2009/0088559 A1
(43) Pub. Date: Apr. 2, 2009

(54) Method for preparing modified polypeptides

(76) Inventors: Torben Halkier, Solroed Strand (DK); Anders Hjelholt Pedersen, Lyngby (DK); Jens Sigurd Okkels, Vedbaek (DK); Kim Vilbour Andersen, Broenshoej (DK)

Correspondence Address: MAXYGEN, INC.;INTELLECTUAL PROPERTY DEPARTMENT, 515 GALVESTON DRIVE, REDWOOD CITY, CA, 94063, US

(21) Appl. No.: 12/011,979

(22) Filed: Jan. 30, 2008

Related U.S. Application Data
(63) Continuation of application No. 10/756,813, filed on Jan. 12, 2004, now abandoned.
Application No. 10/756,813 is a continuation of application No. 10/389,283, filed on Mar. 14, 2003, now abandoned.
Application No. 10/389,283 is a continuation of application No. 10/190,414, filed on Jul. 3, 2002, now abandoned.
Application No. 10/190,414 is a continuation of application No. 09/611,234, filed on Jul. 6, 2000, now abandoned.
(60) Provisional application No. 60/160,693, filed on Oct. 21, 1999.
(60) Provisional application No. 60/189,503, filed on Mar. 15, 2000.
(60) Provisional application No. 60/207,793, filed on May 30, 2000.

(30) Foreign Application Priority Data
Jul. 7, 1999 (DK) PA 1999 00988
Aug. 27, 1999 (DK) PA 1999 01196
Mar. 2, 2000 (DK) PA 2000 00339
May 18, 2000 (DK) PA 2000 00804

(51) Int. Cl. C07K 14/00

(52) U.S. Cl. 530/402

(57) ABSTRACT

Methods for producing polypeptide with altered immunogenicity or improved stability properties are disclosed. The methods involve
a) expressing a diversified population of nucleotide sequences encoding a polypeptide of interest,
b) screening the polypeptides expressed in step a) for function, immunogenicity and/or stability,
c) selecting functional polypeptides having altered immunogenicity and/or increased stability, e.g. functional in vivo half-life as compared to the polypeptide of interest, and
d) optionally subjecting the nucleotide sequence encoding the polypeptide selected in step c) to one or more repeated cycles of steps a)-c). In a further step the expressed polypeptides of step a) or c) can be conjugated to at least one non-polypeptide moiety.

### FIELD OF THE INVENTION

The present invention relates to methods for improving properties of a polypeptide of interest, in particular for altering the immunogenicity and/or increasing the functional in vivo half-life of a polypeptide of interest.

### BACKGROUND OF THE INVENTION

Polypeptides, including proteins, are used for a wide range of applications, including industrial uses and therapeutic applications. A known drawback associated with the use of polypeptides for applications involving contact with humans or animals is that the polypeptides often give rise to an immune response.

Attempts have been made to reduce the immunogenicity and/or allergenicity of polypeptides. One of the most widespread strategies has been to shield epitopes of the polypeptide (which give rise to the undesired immune or allergic response) with polymer molecules, such as polyethylene glycol (PEG), conjugated to the polypeptide. The conjugation of the PEG polymer to a polypeptide is often termed PEGylation. An example of this is disclosed in U.S. Pat. No. 5,856,451 wherein modified polypeptides with reduced allergenicity are disclosed, which polypeptides comprises a parent polypeptide with a molecular weight in the range of 10-100 KDa conjugated to a polymer with a molecular weight in the range of 1-60 KDa. It is stated that the polypeptide to be modified may be a variant of a parent enzyme that has additional attachment groups, such as amino groups not present in the parental enzyme. WO 96/40792 discloses a specific method of PEGylating proteins with a view to reducing allergenicity and/or immunogenicity. WO 97/30148 discloses a method of reducing allergenicity of a protein, wherein the protein is conjugated to at least two polymer molecules. It has been suggested to selectively modify PEGylation attachment groups of polypeptides to be PEGylated. For instance, WO 98/35026 discloses polypeptide-polymer conjugates that have added and/or removed one or more selected attachment groups for coupling polymer molecules on the surface of the three dimensional structure of the polypeptide. By use of site-directed mutagenesis it is suggested to add attachment groups for the polymer molecules at predetermined locations of the polypeptide surface in an attempt to increase the number of polymer molecules, which may be attached and/or to remove attachment groups at or close to the active site of the polypeptide allegedly to avoid excessive PEGylation near the active site, which may lead to decreased activity of the polypeptide.

Another method of modifying polypeptides is disclosed in WO 92/10755 in which it has been suggested to reduce the allergenicity of proteins by identification of epitopes and subsequent destruction of the epitope by modification of amino acid residues constituting the epitope.

U.S. Pat. No. 5,218,092 discloses polypeptides with at least one new or additional carbohydrate attached thereto, the polypeptides allegedly having increased stability as compared to the corresponding unmodified polypeptide. The additional carbohydrate molecule(s) is/are provided by adding one or more additional N-glycosylation sites to the polypeptide backbone, and expressing the polypeptide in a glycosylating host cell. WO 00/26354 discloses a method of reducing allergenicity of proteins, in particular enzymes, wherein the reduction in allergenicity is mediated by increasing the glycosylation of the protein through one or more additional glycosylation sites.

Apart from giving rise to an immune response a further known disadvantage associated with the use of polypeptide-based drugs is that these drugs often are rapidly degraded or eliminated in the body. It has been reported that conjugation of the polypeptide with polymer molecules may increase the functional in vivo half-life. For instance U.S. Pat. No. 4,935,465 discloses a prolonged clearance time of a PEGylated polypeptide due to the increased size of the PEG conjugate of the polypeptide in question. WO 98/48837 relates to single-chain antigen-binding polypeptide-polyalkylene oxide conjugates with reduced antigenicity and increased half-life in the blood stream. The single chain antigen-binding polypeptide to be modified may include one or more inserted Cys or Lys capable of polyalkylene oxide conjugation at certain predetermined sites. Delgado et al., Critical Reviews in Therapeutic Drug Carrier Systems, 9(3,4): 249-304 (1992) is a review article disclosing the state of the art in relation to the uses and properties of PEG-linked polypeptides.

WO 96/12505 discloses conjugates of a polypeptide with a low molecular weight lipophilic compound, which are reported to have improved pharmacological properties. It has been reported that PEGylation of polypeptides may result in reduced function of the polypeptide. Shielding the active site of the polypeptide during PEGylation has been suggested in an attempt to avoid this reduction in activity. More specifically, WO 94/13322 discloses a process for the preparation of a conjugate between a polymer and a first substance having a biological activity mediated by a domain thereof, wherein, during conjugation, the domain of the first substance is protected by a second substance which is removed after conjugation has taken place. It is stated that by using the method the biological activity of the first substance is fully preserved in contrast to the conventional conjugation processes, which normally lead to polymer conjugates with reduced biological activity.

WO 93/15189 relates to a method of preparing proteolytic enzyme-PEG adducts in which the proteolytic enzyme is linked to a macromolecularised inhibitor when reacted with PEG so as to block the active site of the enzyme and thereby preventing that PEG is bound at or near the active site.

WO 97/11957 discloses a process for improving the in vivo function of a polypeptide, in particular factor VIII, by shielding exposed targets of said polypeptide, in which method the polypeptide is immobilized by interaction with a group-specific adsorbent carrying ligands manufactured by organic-chemical synthesis, a biocompatible polymer is activated and conjugated to the immobilized polypeptide and the conjugate is eluted from the adsorbent.

WO97/47751 discloses various forms for modification of a DNAse, e.g. by conjugation to a polymer, a sugar moiety or an organic derivatizing agent. WO 99/40198 discloses various staphylokinase variants modified so as to result in reduced immunogenicity. U.S. Pat. No. 4,904,584 discloses PEGylated lysine depleted polypeptides, wherein at least one lysine residue has been deleted or replaced with any other amino acid residue. WO 99/67291 discloses a process for conjugating a protein with PEG, wherein at least one amino acid residue on the protein is deleted and the protein is contacted with PEG under conditions sufficient to conjugate the PEG to the protein. WO 99/03887 discloses PEGylated variants of polypeptides belonging to the growth hormone superfamily, wherein a cysteine residue has been substituted for a non-essential amino acid residue located in a specified region of the polypeptide.

All of the above described prior art methods are based on using a directed mutagenesis approach to modify polypeptides of interest. Using such site directed mutagenesis techniques, polymer attachment groups are added or removed, thereby enabling construction of polypeptide-polymer conjugates wherein the polymer molecules are attached at certain predetermined locations, typically at the surface of the polypeptide to be modified.

WO 98/27230 discloses the use of shuffling techniques for modifying proteins. The present invention elucidates further methods for modifying polypeptides of interest to have polymer attachment sites that improve one or more functional aspect of the polypeptide.

### BRIEF DISCLOSURE OF THE INVENTION

Rather than introducing attachment groups at predetermined locations at the surface of the polypeptide to be modified, the present invention involves the intelligent creation of diversity in combination with a high throughput screening system.

Accordingly, in a first aspect, the invention relates to a method for altering, i.e., reducing or increasing, the immunogenicity and/or increasing the stability, e.g., functional in vivo half-life, of a polypeptide of interest while maintaining a measurable function of the polypeptide. Such method involves, a) selecting a region of the nucleotide sequence encoding the polypeptide, b) diversifying the selected region, c) expressing the polypeptides encoded by the diversified population of nucleotide sequences, d) conjugating a non-polypeptide moiety to the expressed polypeptides, and e) selecting polypeptide conjugates with altered immunogenicity and/or increased stability.

In some embodiments, the region is selected by computer assisted modeling based on the primary and/or tertiary structure of the polypeptide, e.g. as out-lined in further detail in the section entitled “Strategies for preparing a diversified population of nucleotide sequences”. In some embodiments, diversification is achieved by one or more of DNA shuffling, random mutagenesis, focused mutagenesis, and localized mutagenesis. In some cases, the diversification process involves doping or spiking with oligonucleotides. Optionally, the diversification process is performed recursively. If desired, one or more such diversified nucleotide sequence is further modified by site specific mutagenesis.

In some embodiments, the diversified population of nucleotide sequences includes sequences with altered numbers of codons encoding amino acid residues capable of functioning as attachment groups for non-polypeptide moieties such as sugar moieties, lipophilic molecules, polymer molecules, or organic derivatizing agents.

In preferred embodiments, polynucleotide sequences encoding polypeptides with altered immunogenicity and/or increased stability are identified by a high throughput screening method. For example, a screening assay performed in microtiter plates, on one or more filters or membranes, or pin or bead array, or in a microfluidic device. Another aspect of the invention relates to the production of polypeptides with altered glycosylation patterns having desired properties. In a general embodiment, methods involve a) expressing a diversified population of nucleotide sequences encoding a polypeptide of interest, b) glycosylating the expressed polypeptides, and c) selecting at least one polypeptide with a desired property.

In some embodiments, the population of nucleotide sequences is produced by one or more of DNA shuffling, random mutagenesis, focused mutagenesis, localized mutagenesis, and site specific mutagenesis. In preferred embodiments, the population of nucleotide sequences so produced includes nucleotide sequences encoding polypeptides with altered numbers or locations of glycosylation sites.

Nucleotide sequences encoding polypeptides with desired properties are identified by high throughput screening assays in some embodiments. In some embodiments, the desired property is selected from reduced or increased immunogenicity or increased stability, e.g., increased functional in vivo half-life.

In another aspect, the invention provides methods for altering immunogenicity or improving stability of a polypeptide by a) expressing a diversified population of nucleotide sequences encoding a polypeptide of interest, b) blocking functional sites of the polypeptides with helper molecules, c) conjugating one or more non-polypeptide moieties to the blocked polypeptides, and d) identifying polypeptides with altered immunogenicity or increased stability.

Another method for altering, i.e., reducing or increasing, immunogenicity and/or increasing stability, e.g. functional in vivo half-life of a polypeptide of interest while maintaining a measurable function of the polypeptide involves the basic technical steps of the present invention, i.e.
a) expressing a diversified population of nucleotide sequences encoding a polypeptide of interest,
b) screening the polypeptides expressed in step a) for function, immunogenicity and/or stability,
c) selecting functional polypeptides having altered immunogenicity and/or increased stability, e.g. functional in vivo half-life as compared to the polypeptide of interest, and
d) optionally subjecting the nucleotide sequence encoding the polypeptide selected in step to one or more repeated cycles of steps a)-c).

Yet another method for altering, i.e. reducing or increasing, immunogenicity and/or stability, e.g. functional in vivo half-life, of a polypeptide of interest while maintaining a measurable function of the polypeptide, involves
a) expressing a diversified population of nucleotide sequences encoding a polypeptide of interest,
b) conjugating one or more non-polypeptide moieties to the polypeptides expressed in step a),
c) screening the resulting polypeptide conjugates for function, immunogenicity and/or stability,
d) selecting functional polypeptide conjugates having altered immunogenicity and/or increased stability, e.g. functional in vivo half-life, as compared to the polypeptide of interest, and
e) optionally subjecting the nucleotide sequence encoding the polypeptide part of a polypeptide conjugate selected in step d) to one or more repeated cycles of steps a)-d).

A still further method of constructing a functional polypeptide conjugate having altered immunogenicity and/or increased stability, e.g. functional in vivo half-life, relative to a polypeptide of interest comprises
a) expressing a diversified population of nucleotide sequences encoding the polypeptide of interest,
b) optionally conjugating one or more non-polypeptide moieties to the polypeptides expressed in step a),
c) screening the polypeptides expressed in step a) or, if made the polypeptide conjugates prepared in step b) for function, immunogenicity and/or stability,
d) selecting functional polypeptides or, if made, polypeptide conjugates having altered immunogenicity and/or increased stability, e.g. functional in vivo half-life, as compared to the polypeptide of interest, and
e) optionally subjecting the nucleotide sequence encoding the polypeptide or, if relevant, the polypeptide part of a polypeptide conjugate selected in d) to one or more repeated cycles of steps a)-d).

In still further aspects, the invention relates to a method for constructing a polypeptide conjugate with altered immunogenicity and/or increased stability, e.g. functional in vivo half-life, relative to a polypeptide of interest, which method comprises
a) conjugating one or more non-polypeptide moieties to a polypeptide molecule expressed from a diversified population of nucleotide sequences encoding the polypeptide of interest,
b) screening the resulting polypeptide conjugates for function, immunogenicity and/or stability,
c) selecting functional polypeptide conjugates having altered immunogenicity and/or stability, e.g. increased functional in vivo half-life, relative to the polypeptide of interest, and
d) optionally subjecting the nucleotide sequence encoding the polypeptide part of a polypeptide conjugate selected in c) to one or more repeated cycles of steps a)-c).

In some embodiments, diversification is achieved by one or more of DNA shuffling, random mutagenesis, focused mutagenesis, and localized mutagenesis, as described in the section below entitled “Methods for creating a diversified population of nucleotide sequences”. In some cases, the diversification process involves doping or spiking with oligonucleotides, e.g. as described in said same section. Optionally, the diversification process is performed recursively. If desired, one or more such diversified nucleotide sequence is further modified by site specific mutagenesis, e.g. in order to introduce or remove attachment groups for the non-polypeptide moiety of choice and thereby optimise the overall conjugation pattern of the polypeptide conjugate. Any of the methods of the invention may be conducted in microtiter plates or other available high throughput format, and offer an efficient, and thus attractive, solution for constructing functional polypeptides with altered immunogenicity and/or increased stability properties. In still further aspects, the invention relates to methods for preparing a polypeptide conjugate identified on the basis of any of the above-described methods.

### DETAILED DISCLOSURE OF THE INVENTION

The present invention offers an attractive solution to the problem of altering immunogenicity and/or increasing stability, e.g. functional in vivo half-life of polypeptides of interest. The solution provided by the present invention involves creating and selecting polypeptides with such improved properties, conveniently by use of a high throughput system. The possibility of creating and screening a large number of different polypeptides in a short time makes it possible to search several orders of magnitude more polypeptides than was possible by previously known approaches. Accordingly, the invention enhances the chance of finding the optimal variant from the thousands or ten thousands of variants that may be produced.

The present invention is broadly applicable for the modification of the primary structure of a wide range of polypeptides. Furthermore, the methods apply to conjugation of modified polypeptides with a wide range of non-polypeptide moieties, in particular non-polypeptide moieties that are useful for altering, i.e. decreasing or increasing, immunogenicity and/or increasing stability, e.g. functional in vivo half-life, while maintaining function of the polypeptide of interest. In the present application, emphasis is placed on conjugation to non-polypeptide moieties such as polymers, lipophilic compounds, sugar moieties and organic derivatizing agents. However, it will be understood that the invention can be applied to other types of polypeptide conjugates as well—the only limitation being that the polypeptide can be conjugated to the non-polypeptide moiety of choice (either directly or through a suitable linker) and that the resulting polypeptide conjugate, in addition to the improved properties, is functional. It is intended that methods of preparing such other conjugates are included in the scope of protection afforded by the claims. Similarly, emphasis has been placed on constructing polypeptide conjugates with altered immunogenicity and/or increased functional in vivo half-life. However, it will be understood that the methods of the invention will be useful for constructing polypeptides with other improved properties, the only limitation being that the property to be improved is measurable. Thus, the present claims are also intended to cover the improvement of polypeptides with respect to such other properties.

Modification of polypeptides in accordance with a method of the present invention offers a number of advantages. In addition, or as an alternative, to the improved properties mentioned above (i.e., altered immunogenicity and/or increased functional in vivo half-life) in some instances, in particular when using the methods of the invention involving conjugation to a polymer, a sugar moiety and/or a lipophilic compound, one or more of the following properties can result: cell penetration capability is enhanced, the conjugate is protected from proteolytic digestion and subsequent abolition of activity; affinity for endogenous transport systems is improved, chemical stability against stomach acidity is improved, the function of the polypeptide is improved, e.g., the affinity towards specific surfaces is improved.

### DEFINITIONS

In the context of the present application and invention the following definitions apply:

The term “polypeptide conjugate” or “conjugate” is intended to indicate a chimeric (i.e. heterogeneous (in the sense of composite)) molecule formed by the covalent attachment of one or more polypeptide(s) to one or more non-polypeptide moieties such as polymer molecules, lipophilic compounds, sugar moieties or organic derivatizing agents. The term covalent attachment includes that the specified moieties are either directly covalently joined to one another, or else are indirectly covalently joined to one another through an intervening moiety or moieties, such as a bridge, spacer, or linkage moiety or moieties. Preferably, the chimeric molecule is soluble, such as water soluble, at relevant concentrations, i.e. soluble in physiological fluids such as blood. The term “non-conjugated polypeptide” is used about the polypeptide part of the conjugate. Preferred examples of a conjugate of the invention include a glycosylated polypeptide and a PEGylated polypeptide.

The term “non-polypeptide moiety” is intended to indicate a molecule, different from a peptide polymer composed of amino acid monomers and linked together by peptide bonds (except where the polymer is human albumin or another abundant plasma protein), which molecule is capable of conjugating to an attachment group of the polypeptide of the invention. The term “polymer molecule” is defined as a molecule formed by covalent linkage of two or more monomers. The term “polymer” may be used interchangeably with the term “polymer molecule”. Except where the number of polymer molecule(s) in the conjugate is expressly indicated every reference to “a polymer”, “a polymer molecule”, “the polymer” or “the polymer molecule” contained in a conjugate or otherwise used in a method of the present invention shall be a reference to one or more polymer molecule(s) in the conjugate.

The term “sugar moiety” is intended to indicate a carbohydrate-containing molecule comprising one or more monosaccharide residues, capable of being attached to the polypeptide (to produce a polypeptide conjugate in the form of a glycosylated polypeptide) by way of in vivo or in vitro glycosylation. The term “in vivo glycosylation” is intended to mean any attachment of a sugar moiety occurring in vivo, i.e. during posttranslational processing in a glycosylating cell used for expression of the polypeptide, e.g. by way of N-linked and O-linked glycosylation. Usually, the N-glycosylated sugar moiety has a common basic core structure composed of five monosaccharide residues, namely two N-acetylglucosamine residues and three mannose residues. The exact sugar structure depends, to a large extent, on the glycosylating organism in question and on the specific polypeptide. Depending on the host cell in question the glycosylation is classified as a high mannose type, a complex type or a hybrid type. The term “in vitro glycosylation” is intended to refer to a synthetic glycosylation performed in vitro, normally involving covalently linking a sugar moiety to an attachment group of a polypeptide, optionally using a cross-linking agent. In vivo and in vitro glycosylation are discussed in detail further below. Alternative terms to sugar moiety include carbohydrate moiety, carbohydrate chain, oligosaccharide moiety or oligosaccharide chain.

The term “attachment group” is intended to indicate an amino acid residue group capable of coupling to a non-polypeptide moiety such as a polymer molecule, a lipophilic compound, a sugar moiety or an organic derivatizing agent suitable for use in the construction of a polypeptide conjugate by a method of the invention. For polymer conjugation, a frequently used attachment group is the ε-amino group of lysine. Another attachment group is the N-terminal amino group of the polypeptide. Polymer molecules may also be coupled to free carboxylic acid groups, suitably activated carbonyl groups, oxidized carbohydrate moieties and mercapto groups. For instance, polymer attachment groups may be constituted by the carboxylic acid groups (—COOH) of amino acid residues in the polypeptide chain. Carboxylic acid polymer attachment groups may be the carboxylic acid group of aspartate or glutamate and the C-terminal COOH-group of the polypeptide. The sulfhydryl group of free Cys can be derivatized using, e.g., PEG-vinylsulphone. For conjugation to a lipophilic compound the following polypeptide groups may function as attachment groups: the N-terminal or C-terminal of the polypeptide, the hydroxy groups of the amino acid residues Ser, Thr or Tyr, the E-amino group of Lys, the SH group of Cys or the carboxyl group of Asp and Glu.

For in vivo N-glycosylation, the term “attachment group” is used in an unconventional way to indicate the amino acid residues constituting an N-glycosylation site (with the sequence N—X′-S/T/C—X″, wherein X′ is any amino acid residue except proline, X″ any amino acid residue that may or may not be identical to X′ and preferably is different from proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site are present. Accordingly, when the non-polypeptide moiety is a sugar moiety and the conjugation is to be achieved by N-glycosylation, the term “amino acid residue comprising an attachment group for the non-polypeptide moiety” as used in connection with alterations of the amino acid sequence of the parent polypeptide is to be understood as amino acid residues constituting an N-glycosylation site are to be altered in such a manner that either a functional N-glycosylation site is introduced into the amino acid sequence or removed from said sequence. An “O-glycosylation site” is the OH-group of a serine or threonine residue. For in vitro glycosylation useful attachment groups include those of arginine, histidine, a free carboxyl group, a free sulfhydryl group such as that of cysteine, a free hydroxyl group such as that of serine, threonine, or hydroxyproline, an aromatic residue such as that of phenylalanine, tyrosine or tryptophan or the amide group of glutamine. For coupling to an organic derivatizing agent an attachment group is typically N- or C-terminal residues, cysteine, histidine, lysine, arginine, aspartic acid or glutamic acid.

In the present application, amino acid names and atom names (e.g. CA, CB, NZ, N, O, C, etc) are used as defined by the Protein DataBank (PDB) (www.pdb.org) which are based on the IUPAC nomenclature (IUPAC Nomenclature and Symbolism for Amino Acids and Peptides (residue names, atom names e.t.c.), Eur. J. Biochem., 138, 9-37 (1984) together with their corrections in Eur. J. Biochem., 152, 1 (1985). CA is sometimes referred to as Cα, CB as Cβ. The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/mutations is typically A15 (indicates an alanine residue in position 15 of the polypeptide), A15T (indicates replacement of the alanine residue in position 15 with a threonine residue), A15T,S (indicates replacement of the alanine residue in position 15 with a threonine residue or a serine residue). Multiple substitutions are indicated with a “+”, e.g. A15T+F57S means an amino acid sequence which comprises a substitution of the alanine residue in position 15 for a threonine residue and a substitution of the phenylalanine residue in position 57 for a serine residue.

The term “diversified population of nucleotide sequences encoding a polypeptide of interest” is intended to indicate ten or more nucleotide sequences, preferably at least 500, such as at least 1000 nucleotide sequences, which differ from each other in one or more nucleotides (thereby providing diversity), which population is capable of expressing a polypeptide which has one or more of the same functions as the polypeptide of interest (such as a biological function), and, in addition, one or more modified properties (such as a different conjugation behavior, e.g., a different glycosylation pattern or differences in attachment group for a polymer or a lipophilic compound). In this context the term “same function” should be understood qualitatively, and not necessarily quantitatively. Since a critical element of the methods of the invention is the diversity of the population of nucleotide sequences, it will be understood that the exact identity of each of the nucleotide sequences constituting the diversified population is not important as long as the population contains nucleotide sequences encoding a polypeptide with relevant function(s) (which will be evident when conducting the screening and selection steps of a method of the invention). Accordingly, the term “encoding a polypeptide of interest” as used in the context of the diversified population of nucleotide sequences is intended to indicate that some, but normally far from all of the nucleotide sequences of the diversified population encode a polypeptide exhibiting one or more of the same functions as the polypeptide of interest. The polypeptides encoded by the diversified population and exhibiting one or more of the same functions as the polypeptide of interest is, e.g., identical to the polypeptide of interest or a variant thereof, i.e., differing in one or more amino acid residues as compared to the polypeptide of interest.

Typically, the diversified population is provided in the form of a nucleotide sequence library comprising nucleotide sequences which are created by random mutagenesis of a nucleotide sequence encoding the polypeptide of interest, or is the result of shuffling, e.g., between two or more homologous nucleotide sequences which are homologous to a nucleotide sequence encoding the polypeptide of interest and which themselves are sometimes created by random or site-directed mutagenesis of a nucleotide sequence encoding the polypeptide of interest. Normally, a main part, such as at least 20%, typically at least 30% or at least 40%, more typically at least 50% or at least 60%, even more typically at least 70% or at least 80% of the nucleotide sequences display a nucleotide sequence identity of at least 40% identity, such as at least 50% or 60% identity, in particular at least 70% identity to each other.

The term “random mutagenesis” refers to a mutagenic process that is random with respect to the site of mutation within the subject nucleic acid, and that is random with respect to the mutations introduced, e.g., chemical mutagenesis, uv or γ irradiation, passage through repair deficient cells, etc. The term “localized mutagenesis” is used to indicate that the mutagenic process occurs preferentially in a predetermined portion or subsequence of the subject nucleic acid. “Focused mutagenesis” refers to a mutagenic process that is biased with respect to the mutations produced, e.g., by codon preference, or oligonucleotide doping or spiking. In the context of the present invention, “site directed mutagenesis” refers to an alteration at a predetermined nucleotide position or positions, normally with the aim of altering one or more amino acid residues of the encoded amino acid sequence. The site-directed mutagenesis is normally designed on the basis of an analysis of a primary or tertiary (e.g. model) structure of the polypeptide to be modified.

The term “nucleotide sequence” is intended to indicate a consecutive stretch of two or more nucleotides molecules. The nucleotide sequence can be of genomic, cDNA, RNA, semisynthetic, synthetic origin, or any combinations thereof.

The “homology” or “identity” as used in connection with nucleotide or amino acid sequences is used in its conventional meaning. Amino acid sequence homology/identity is conveniently determined from aligned sequences (aligned by use of the CLUSTALW, version 1.74 using default parameters or provided from the PFAM families database version 4.0 (see Materials and Methods) by use of GENEDOC version 2.5 (Nicholas, K. B., Nicholas H. B. Jr., and Deerfield, D. W. II. 1997 GeneDoc: Analysis and Visualization of Genetic Variation, EMBNEW.NEWS 4:14; Nicholas, K. B. and Nicholas H. B. Jr. 1997 GeneDoc: Analysis and Visualization of Genetic Variation). Nucleotide sequence homology/identity is determined using the AlignX programme of the Vector NTI package available from Informax Inc.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell. “Vector” refers to a plasmid or other nucleotide sequences that are capable of replicating within a host cell or being integrated into the host cell genome, and as such, are useful for performing different functions in conjunction with compatible host cells (a vector-host system): to facilitate the cloning of the nucleotide sequence of interest, i.e. to produce usable quantities of the sequence, to direct the expression of the gene product encoded by the sequence and to integrate the nucleotide sequence of interest into the genome of the host cell. The vector will contain different components depending upon the function it is to perform.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is position so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “introduce” is intended to include substitution of an existing amino acid residue and insertion of additional amino acid residue. The term “remove” is intended to include substitution of the amino acid residue to be removed with another amino acid residue and deletion (without substitution) of the amino acid residue to be removed.

The term “immunogenicity” as used in connection with a given substance is intended to indicate the ability of the substance to induce a response from the immune system. Immune responses include both cell and antibody mediated responses. A substance which is capable of giving rise to an immune response may be called an immunogen (i.e., a substance which, when introduced into the circulatory system of a human or animal is capable of directly or indirectly stimulating an immunological response resulting in the formation of immunoglobulins or specific T-cells), an antigen (i.e., a substance which by itself is capable of generating antibodies when recognized as a non-self molecule and which is recognized by an antibody or T-cell receptor), or an allergen (i.e., an antigen which may give rise to allergic sensitization or an allergic response, e.g., by IgE antibodies in humans). See, e.g., Roitt: Essential Immunology (8^{th} Edition, Blackwell) for further definition of immunogenicity.

The term “altered immunogenicity” is intended to indicate that the polypeptide conjugate produced by a method of the present invention gives rise to a measurably lower altered, either reduced or increased, immune response than the polypeptide of interest as determined under comparable conditions.

The term “functional in vivo half-life” is used in its normal meaning, i.e. the time in which 50% of a given function (such as biological or catalytic activity) of the conjugate is retained, when tested in vivo, or in which half of the polypeptide conjugate molecules circulate in the plasma or bloodstream prior to being cleared, normally by the action of one or more of the reticuloendothelial systems (RES), kidney, spleen or liver, or by specific or unspecific proteolysis. Clearance depends on size (e.g. molecular weight or hydrodynamic volume) relative to the cutoff for glomerular filtration, shape/rigidity, charge, attached sugar chains, and the presence of cellular receptors for the protein. Alternative terms to “functional in vivo half-life” are “plasma clearance”, “serum half-life”, and “in vivo half-life”. It will be understood that functional in vivo half-life is of particular interest for pharmaceutical or veterinary polypeptides.

The term “increased functional in vivo half-life” is used to indicate that the functional in vivo half-life of the polypeptide conjugate is statistically significantly increased relative to that of the unconjugated polypeptide of interest as determined under comparable conditions. For instance, the functional in vivo half-life is increased at least 2 times, such as at least 10 times or at least 100 times as compared to that of the unconjugated polypeptide.

The term “function” is intended to indicate one or more specific functions of the polypeptide of interest. Typically, a given polypeptide has many different functions, examples of which are given further below in the section entitled “Screening for function”. Since methods of the present invention are generally applicable to polypeptides it will be understood that the meaning of the term has to be related to the polypeptide of interest. The term “function” is to be understood qualitatively (i.e., having a similar function as the polypeptide of interest) and not necessarily quantitatively (i.e., the magnitude of the function is not necessarily similar).

The term “stability” is used with respect to the polypeptide's capability of resisting degradation or elimination when present in a relevant environment, e.g. under conditions of storage or use in vitro or in vivo. Examples of properties of relevance for stability include stability towards proteolytic degradation, pH, temperature, certain chemicals, resistance to glomerular filtration, etc. An “improved” or “increased” stability can, e.g. be measured in terms of functional in vivo half-life, plasma half life, shelf life (in particular for industrial enzymes), etc., the specific parameter to be chosen usually depending on the environment in which the polypeptide is to used.

The term “measurable function” or “functional polypeptide” is intended to indicate that the modified polypeptide resulting from the method of the invention has preserved a sufficiently high function of interest to make it possible to measure the function by standard methods known in the art. In this context, the term “measurable” should be considered in relation to the specific use of the polypeptide of interest. For instance, if the polypeptide is a hormone and the function of interest is the hormone's affinity towards a specific receptor a measurable function is defined to be an observable affinity between the hormone and the receptor as determined by the normal methods used for measuring such affinity. If the polypeptide is an enzyme and a function of interest is the enzyme activity a measurable function is the enzyme's ability to catalyze a reaction involving the normal substrates of the enzyme as measured by the normal methods for determining the enzyme activity in question. It will be understood that the magnitude of a “measurable function” is related to the polypeptide of interest and thus may vary considerably among different polypeptides. Normally, a measurable function is at least 1%, such as at least 5% of the function of the unmodified or non-conjugated polypeptide of interest, such as at least 10% as measured under comparable conditions. Preferably, a measurably function is 15%, such as at least 25%, in particular at least 40% and more preferably at least 50% of the function of the unmodified or non-conjugated polypeptide of interest as measured under comparable conditions. Most preferably, a measurable function is at least 60%, such as at least 75%, in particular at least 80% or at least 95% of the function of the unmodified or non-conjugated polypeptide of at interest. In certain cases the measurable function is at least 100% such as at least 120% of the unmodified or non-conjugated polypeptide of interest as determined under comparable conditions. In the present context the term “functional site” is intended to indicate one or more amino acid residues which is/are essential for or otherwise involved in the function or performance of the polypeptide, i.e., the amino acid residues which mediates a desired biological activity of the polypeptide in question. Such amino acid residues are “located at” the functional site. The functional site can be determined by methods known in the art and is preferably identified by analysis of a structure of the polypeptide complexed to a relevant ligand. For instance, the functional site can be a binding site, a catalytic site, a regulatory site, or an interaction site. The polypeptide of interest can have one or more functional sites. For instance, when the polypeptide is an enzyme a functional site comprises the amino acid residues making up the catalytic site, e.g., the catalytic triad of serine proteases, and/or amino acid residues involved in substrate binding. When the polypeptide is a hormone or a growth factor a functional site is normally a binding site such as a receptor-binding site. Typically, the growth factor or hormone has several binding sites. When the polypeptide is an antibody a functional site is, e.g., an antigen-binding site. Normally, an antibody has two antigen-binding sites. When the polypeptide is a regulatory protein, a typical functional site is an interaction site. When the polypeptide is a receptor a typical functional site is a ligand binding site or a signalling/effector site. When the polypeptide is an enzyme inhibitor a functional site is a site interacting with the functional site of the enzyme.

The term “equivalent position” is intended to indicate a position in the amino acid sequence of a given polypeptide, which is homologous (i.e., corresponding in position in either primary or tertiary structure) to a position in the amino acid sequence of another polypeptide belonging to the same polypeptide sequence family. Where possible, the “equivalent position” is conveniently determined on the basis of an alignment of members of the polypeptide sequence family in question, suitably prepared by using the alignment program CLUSTALW version 1.74, or from the PFAM protein families database (see, Materials and Methods).

The term “polypeptide sequence family” is used in its conventional meaning, i.e., to indicate a group of polypeptides, which are related to each other by function and structure in terms of having an amino acid sequence which exhibits a sufficient degree of homology to allow alignment of the sequences. An alternatively used term is “protein sequence family”. Polypeptide sequence families are available, e.g. from the PFAM families, version 4.0, or can be prepared by use of a suitable computer program such as CLUSTALW version 1.74. The preparation of a polypeptide sequence family is described in further detail in the Materials and Methods section hereinafter.

The term “high throughput screening” is intended to indicate a screening of a large number of samples (such as more than 100 samples per day). The screening can be conducted manually, but is preferably done using an automatized or semi-automatized system.

### Polypeptide of Interest

In the present context the term “polypeptide of interest” is intended to indicate any molecule that comprises a stretch of two or more amino acid residues, typically at least 20 amino acid residues. In addition, the polypeptide of interest can be post-translationally modified and thereby comprise other types of molecules such as sugar moieties (apart from any sugar moieties to which the polypeptide of interest can be conjugated by a method of the present invention). Preferably, the polypeptide of interest is a protein, a glycoprotein or an oligopeptide that contains in the range of 30 to 4500 amino acids, preferably in the range of 40 to 3000 amino acids.

The methods of the present invention are broadly applicable. The polypeptide can be of any origin, including microbial, mammalian, plant and insect origin as long as it is encoded by a nucleotide sequence, which is capable of being modified according to a method of the present invention. For instance, the microbial polypeptide is of fungal, yeast or bacterial origin; the mammalian polypeptide is of human, porcine, ovine, urcine, murine, rabbit, donkey, or bat origin. Furthermore, the polypeptide can be of snake, leech, frog or mosquito origin. Preferably, the polypeptide of interest is of microbial origin or of human origin.

It will be understood that the term “polypeptide of interest” includes at least the following types of polypeptides:

Native or wild type polypeptides, i.e., polypeptides that can be found in nature; polypeptides which have been prepared by genetic or other modification of a native or wildtype polypeptide (e.g., by substitution, deletion or truncation of one or more amino acid residues of the polypeptide or by addition or insertion of one or more amino acid residues into the polypeptide) so as to modify the amino acid sequence constituting said native or wildtype polypeptide, e.g., by modification of a polynucleotide encoding the polypeptide of interest. This polypeptide type is also termed “a variant”;
polypeptides, which for other reasons are different from those found in nature.

The polypeptide of interest can be a pharmaceutical or veterinary polypeptide, i.e., a polypeptide that is physiologically active when introduced into the circulatory system of or otherwise administered to a human or an animal, or a diagnostic polypeptide intended for use in diagnosis. Furthermore, the polypeptide of interest can be an industrial polypeptide intended for industrial uses such as e.g., in the manufacture of goods wherein the polypeptide constitutes a functional ingredient or wherein the polypeptide is used for processing or other modification of raw ingredients during the manufacturing process. The industrial polypeptide is typically an enzyme and can be used in products or in the manufacture of products such as detergents, household articles, personal care products, agrochemicals, textile, food products, in particular bakery products, feed products, or in industrial processes such as hard surface cleaning. The industrial polypeptide is normally not intended for internal administration to humans or animals.

For example, the polypeptide of interest is an antibody or antibody fragment, immunoglobulin or immunoglobulin fragment, a plasma protein, an erythrocyte or thrombocyte protein, a cytokine, a growth factor, a binding protein, a profibrinolytic protein, a protease inhibitor, an antigen, an enzyme, a ligand, a receptor, or a hormone. In the present context, the term “antibody” includes single monoclonal antibodies (including agonist and antagonist antibodies) and antibody compositions with polyepitopic specificity that can also be termed polyclonal antibodies. The term “monoclonal antibody” is used in its conventional meaning to indicate a population of antibodies of substantially homogeneous antibodies. The individual antibodies comprised in the population have identical binding affinities and vary structurally only to a limited extent. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. Preferred antibody targets for the present invention are human or humanized monoclonal antibodies.

“Antibody fragment” is defined as a portion of an intact antibody comprising the antigen binding site or the entire or part of the variable region of the intact antibody, wherein the portion is free of the constant heavy chain domains (i.e. CH2, CH3, and CH4, depending on antibody isotype) of the Fc regions of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (which can also be termed a single chain antibody fragment or a single chain polypeptide).

Immunoglobulins of interest include IgG, IgE, IgM, IgA, IgD and fragments thereof. More specifically, the polypeptide of interest can be i) a plasma protein, e.g. a factor from the coagulation system, such as Factor VII, Factor VIII, Factor IX, Factor X, Factor XIII, thrombin, protein C, antithrombin III or heparin co-factor II, a factor from the fibrinolytic system such as pro-urokinase, urokinase, tissue plasminogen activator, plasminogen activator inhibitor 1 (PAI-1) or plasminogen activator inhibitor 2 (PAI-2), the Von Willebrand factor, or an α-1-proteinase inhibitor, ii) a erythrocyte or thrombocyte protein, e.g. hemoglobin, thrombospondin or platelet factor 4, iii) a cytokine, e.g. an interleukin such as interleukin (IL) 1, IL-2, IL-4, IL-5, IL-6, IL-9, IL-10, IL-11, IL-12, IL-13, IL-15, IL-16, IL-17, IL-18, or IL-IRa, an interferon such as interferon-α, interferon-β or interferon-γ, a colony-stimulating factor such as GM-CSF or G-CSF, stem cell factor (SCF), a member of the tumor necrosis factor family (e.g TNF-α, lymphotoxin-α, lymphotoxin-β, FasL, CD40L, CD30L, CD27L, Ox40L, 4-1BBL, RANKL, TRAIL, TWEAK, LIGHT, TRANCE, APRIL, THANK or TALL-1), iv) a growth factor, e.g platelet-derived growth factor (PDGF), transforming growth factor α (TGF-α), transforming growth factor β (TGF-β), epidermal growth factor (EGF), vascular endothelial growth factor (VEGF), somatotropin (growth hormone), a somatomedin such as insulin-like growth factor I (IGF-I) or insulin-like growth factor II (IGF-II), erythropoietin (EPO), thrombopoietin (TPO) or angiopoietin, v) a profibrinolytic protein, e.g. staphylokinase or streptokinase, vi) a protease inhibitor, e.g. aprotinin or CI-2A, vii) an enzyme, e.g. superoxide dismutase, catalase, uricase, bilirubin oxidase, trypsin, papain, asparaginase, arginase, arginine deiminase, adenosin deaminase, ribonuclease, alkaline phosphatase, β-glucuronidase, purine nucleoside phosphorylase or batroxobin, viii) an opioid, e.g. endorphins, enkephalins or non-natural opioids, ix) a hormone or neuropeptide, e.g. insulin, calcitonin, glucagons, adrenocorticotropic hormone (ACTH), somatostatin, gastrins, cholecystokinins, parathyroid hormone, luteinizing hormone, gonadotropin-releasing hormone, chorionic gonadotropin, corticotropin-releasing factor, vasopressin, oxytocin, antidiuretic hormones, thyroid-stimulating hormone, thyrotropin-releasing hormone, relaxin, glucagon-like peptide 1 (GLP-1), glucagon-like peptide 2 (GLP-2), prolactin, neuropeptide Y, peptide YY, pancreatic polypeptide, leptin, orexin, CART (cocaine and amphetamine regulated transcript), a CART-related peptide, melanocortins (melanocyte-stimulating hormones), melanin-concentrating hormone, follicle-stimulating hormone, natriuretic peptides, adrenomedullin, endothelin, exendin, secretin, amylin (IAPP; islet amyloid polypeptide precursor), vasoactive intestinal peptide (VIP), pituitary adenylate cyclase activating polypeptide (PACAP), agouti and agouti-related peptides or somatotropin-releasing hormones, or x) another type of protein or peptide such as thymosin, bombesin, bombesin-like peptides, heparin-binding protein, soluble CD4, pigmentary hormones, hypothalamic releasing factor, malanotonins or phospholipase activating protein.

Examples of, in particular industrial, enzymes include hydrolases, such as proteases or lipases, oxidoreductases, such as laccase and peroxidase, transferases such as transglutaminases, isomerases, such as protein disulphide isomerase and glucose isomerase, cell wall degrading enzymes such as cellulases, xylanases, pectinases, mannanases, etc., amylolytic enzymes such as endoamylases, e.g., alpha-amylases, or exo-amylases, e.g., beta-amylases or amyloglucosidases, etc.

### Methods for Creating a Diversified Population of Nucleotide Sequences

The diversified population of nucleotide sequences encoding a polypeptide of interest is prepared by any suitable method known in the art. For example, the diversified population can be prepared by methods involving gene shuffling, other recombination between nucleotide sequences, random, localized or focused mutagenesis or any combination of these methods.

For example, the diversified population of nucleotide sequences can be prepared from two or more nucleotide sequences which are sufficiently homologous to allow recombination between the sequences or parts thereof. For instance, the diversified population of nucleotide sequences is prepared by combination between such sequences or parts thereof. The combination of nucleotide sequences or sequence parts is conveniently conducted by methods known in the art, for instance methods which involve homologous cross-over such as disclosed in U.S. Pat. No. 5,093,257, or methods which involve gene shuffling, i.e., recombination between two or more homologous nucleotide sequences resulting in new nucleotide sequences having a number of nucleotide alterations when compared to the nucleotide sequences used for the recombination. In order for homology based nucleic acid shuffling to take place the nucleotide sequences is preferably at least 50% identical, such as at least 60% identical, more preferably at least 70% identical, such as at least 80% identical. The recombination can be performed in vitro or in vivo. Examples of suitable in vitro gene shuffling methods are disclosed by Stemmer et al (1994), Proc. Natl. Acad. Sci. USA; vol. 91, pp. 10747-10751; Stemmer (1994), Nature, vol. 370, pp. 389-391; Smith (1994), Nature vol. 370, pp. 324-325; Zhao et al., Nat. Biotechnol. 1998, March; 16(3): 258-61; Zhao H. and Arnold, F B, Nucleic Acids Research, 1997, Vol. 25. No. 6 pp. 1307-1308; Shao et al., Nucleic Acids Research 1998, Jan. 15; 26(2): pp. 681-83; and WO 95/17413. Example of a suitable in vivo shuffling method is disclosed in WO 97/07205.

Furthermore, the diversified population of nucleotide sequences can be a randomly mutagenized library, conveniently prepared by subjecting a nucleotide sequence encoding the polypeptide of interest to mutagenesis to create a large number of mutated nucleotide sequences. The mutagenesis can be entirely random, both with respect to where in the nucleotide sequence the mutagenesis occurs and with respect to the nature of mutagenesis. Alternatively, the mutagenesis can be conducted so as to randomly mutate one or more selected regions of the polypeptide (“localized mutagenesis”), and/or directed towards introducing certain types of amino acid residues, in particular amino acid residues containing an attachment group, at random into the polypeptide molecule or at random into one or more selected regions of the polypeptide (“focused mutagenesis”). Besides substitutions, the mutagenesis can also cover random introduction of insertions or deletions. Preferably, the insertions are made in reading frame, e.g., by performing multiple introduction of three nucleotides as described by Hallet et al., Nucleic Acids Res. 1997, 25(9):1866-7 and Sondek and Shrotle, Proc Natl. Acad. Sci USA 1992, 89(8):3581-5.

The random mutagenesis (either of the whole nucleotide sequence or of one or more selected regions of the nucleotide) can be performed by any suitable method. For example, the mutagenesis is performed using a suitable physical or chemical mutagenizing agent, a suitable oligonucleotide, PCR generated mutagenesis or any combination of these mutagenizing agents and/or other methods according to state of the art technology, e.g. as disclosed in WO 97/07202.

Error prone PCR generated mutagenesis, e.g. as described by J. O. Deshler (1992), GATA 9(4): 103-106 and Leung et al., Technique (1989) Vol. 1, No. 1, pp. 11-15, is particularly useful for mutagenesis of longer peptide stretches (corresponding to nucleotide sequences containing more than 100 bp) or entire genes, and are preferably performed under conditions that increase the misincorporation of nucleotides. Mutagenesis based on doped or spiked oligonucleotides or by specific sequence oligonucleotides, is of particular use for mutagenesis of one or more regions containing shorter nucleotide sequences (normally containing less than 100 nucleotides per region). Mutagenesis of several regions is conveniently conducted by spiking with several oligos and combining them by PCR. Doping or spiking with oligonucleotides can also be used for random mutagenesis of nucleotide sequences encoding longer peptide stretches or entire genes when it is desirable to be able to control the random mutagenesis to a higher extent than is possible with error prone PCR generated mutagenesis.

In some embodiments, localized or focused mutagenesis of one or more selected regions of a nucleotide sequence encoding the polypeptide of interest is performed using PCR generated mutagenesis, in which one or more suitable oligonucleotide primers flanking the area to be mutagenized are used. In some cases, doping or spiking with oligonucleotides is used to introduce mutations so as to remove or introduce attachment groups for a non-polypeptide moiety of interest. Preferably, the spiking or doping is designed to avoid the introduction of codons for unwanted amino acid residues (by lowering the amount of or completely avoiding the nucleotides resulting in these codons) or to increase the likelihood that a particular type of amino acid residue (e.g. an amino acid comprising an attachment group for the non-polypeptide moiety of interest) is introduced into a desired position or region of the polypeptide (by increasing the number of codons for the amino acid residue). State of the art knowledge and computer programs (e.g. as described by Siderovski D P and Mak T W, Comput. Biol. Med. (1993) Vol. 23, No. 6, pp. 463-474 and Jensen et al. Nucleic Acids Research, 1998, Vol. 26, No. 3) can be used for calculating the most optimal nucleotide mixture for a given amino acid preference. The oligonucleotides can be incorporated into the nucleotide sequence encoding the polypeptide of interest by any published technique using e.g. PCR, LCR or any DNA polymerase or ligase.

In a preferred embodiment, the mutagenesis is localized to two, three, four, five, six or more regions at the same time by synthesizing random, doped, biased and/or specific oligonucleotides covering each region and assembling the oligonucleotides by state of the art technologies, for example by a PCR method. One convenient PCR method involves a PCR in which the nucleotide sequence encoding the polypeptide of interest is used as a template and, e.g., random, doped, biased and/or specific oligonucleotides are used as primers. In addition, cloning primers localized outside the targeted regions can be used. The resulting PCR product can either directly be cloned into an appropriate expression vector or gel purified and amplified in a second PCR reaction using the cloning primers and cloned into an appropriate expression vector.

In addition to the recombination, shuffling, random, localized and focused mutagenesis methods described herein, it is occasionally useful to employ site specific mutagenesis techniques to modify one or more selected amino acids in a polypeptide of interest in the context of the present diversification and screening methods. Site-specific mutagenesis can be conducted in any part of the polypeptide, e.g. within a region which has already been modified by a method of the invention or outside such region. Site-specific mutagenesis is conveniently designed on the basis of a primary or tertiary (e.g. model structure) of the modified polypeptide or polypeptide conjugate resulting from a method of the invention. The site-specific mutagenesis is normally followed by screening for function and one or more improved properties as described herein.

The nucleotide sequence(s) or nucleotide sequence region(s) to be mutagenized is typically present on a suitable vector such as a plasmid or a bacteriophage, which as such is incubated with or otherwise exposed to the mutagenizing agent. The nucleotide sequence(s) to be mutagenized can also be present in a host cell either by being integrated into the genome of said cell or by being present on a vector harboured in the cell. Alternatively, the nucleotide sequence to be mutagenized is in isolated form. The nucleotide sequence is preferably a DNA sequence such as a cDNA, genomic DNA or synthetic DNA sequence.

Subsequent to the incubation with or exposure to the mutagenizing agent, the mutated nucleotide sequence, normally in amplified form, is expressed by culturing a suitable host cell carrying the nucleotide sequence under conditions allowing expression to take place. The host cell used for this purpose is one, which has been transformed with the mutated nucleotide sequence(s), optionally present on a vector, or one which carried the nucleotide sequence during the mutagenesis, or any kind of gene library. A host cell of choice for screening is one capable of a reasonable transformation frequency such as bacterium, yeast or fungus. Alternatively, a high throughput transfection system of mammalian cells or other cells capable of a desirable post-translational modification can be employed. The latter is of particular interest when post-translational processing is of importance and examples include CHO (Chinese Hamster Ovary) and COS and BHK (Baby Hamster Kidney) cells.

### Strategies for Preparing a Diversified Population of Nucleotide Sequences

An analysis of which amino acid residue(s) or region(s) of the polypeptide of interest constitute(s) preferred target(s) for modification, e.g., by use of localized or focused mutagenesis techniques, is suitably performed as described in the Materials and Methods section herein. Alternatively, such modification is performed randomly as described in further detail hereinafter. The identity of the attachment group to be introduced/removed depends on the identity of the non-polypeptide moiety to which the polypeptide is to be conjugated, e.g. as evident from the “Definitions” section herein.

According to one embodiment of the invention the diversified population of nucleotide sequences is constructed by localizing the random mutagenesis (e.g. to introduce and/or remove amino acid residues comprising attachment group) to one or more defined region(s) of the polypeptide of interest, “localized mutagenesis.” For instance, the mutagenesis is focused by being designed to introduce amino acid residues with an attachment group for a polymer molecule, a lipophilic compound, a sugar moiety or an organic derivatizing agent in one or more specified regions and/or to remove such (or other) residues from one or more other regions of the polypeptide of interest. In the present context, the term “region” is intended to include a single amino acid residue as well as a group of two, three, four or more amino acid residues which are located closely together either in the three-dimensional structure or the primary structure of the polypeptide of interest.

According to one embodiment of the present invention, a region to be selected for localized or focused mutagenesis is a region that can advantageously be enriched in one or more amino acid residues, containing an attachment group for the non-polypeptide moiety in question. For instance, the region is selected from the following group of regions:

A region that contains one or more amino acid residues potentially exposed to the surface of the polypeptide of interest.
A region that contains one or more amino acid residues occupying an equivalent position to a residue in any of the other members of the protein sequence family, which comprises an attachment group (only including those family members having an amino acid sequence which has 40% or higher identity to the given protein amino acid sequence).
A region in which one or more amino acid residues containing an attachment group can be inserted by way of conservative mutation of one or more existing amino acid residues, e.g., to mutate Arg to Lys, Asn to Asp and/or Gln to Glu.
A region in which one or more amino acid residues having their CB (or CA in the case of Gly) at a distance of more than 8 Å, such as more than 10 Å from CB of the attachment group of the nearest amino acid residue containing such group (in order to obtain a balanced distribution of attachment groups at the surface of the polypeptide of interest).
A region wherein one or more amino acid residues having their CB (or CA in the case of Gly) at a distance of more than 10 Å from the attachment group of the nearest amino acid residue containing such group (in order to obtain a balanced distribution of attachment groups at the surface of the polypeptide of interest).
A region that comprises one or more amino acid residues present in a known epitope region (i.e. amino acid residues contributing to an epitope or located in such a manner that conjugation of a non-polypeptide moiety to the amino acid residue shields an epitope), the epitope region, e.g., being identified by epitope mapping.

In particular, it can be of interest to perform localized or focused mutagenesis of amino acid residues in more than one of the above-mentioned regions. For instance, localized or focused mutagenesis is conveniently performed in a region containing amino acid residue(s) potentially exposed at the surface of the polypeptide and that also belongs to one of the other above specified regions.

Furthermore, in accordance with this embodiment, mutagenesis is conducted in one, and preferably two or more part(s) of the nucleotide sequence corresponding to one or more of the above region(s) of the polypeptide of interest to introduce an amino acid residue containing an attachment group into this/these region(s). Preferably, the region(s) to be mutagenized does/do not contain an amino acid residue or contain(s) only few, e.g., one or two amino acid residues having an attachment group. Furthermore, in order to preserve function of the polypeptide of interest it is sometimes desirable that the amino acid residue(s) to be introduced, which contain(s) an attachment group, is/are not introduced at a functional site of the polypeptide of interest.

In order to ensure introduction of amino acid residue(s), e.g., containing an attachment group, focused mutagenesis is used, conveniently designed in such a way that the resulting diversified population of nucleotide sequences is enriched in codons encoding such amino acid residue(s). Preferably, the diversified population of nucleotide sequences is enriched in sequences encoding one or more amino acid residue(s) selected from the group consisting of lysine, arginine, aspartic acid, glutamic acid, tyrosine and cysteine. For example, when the non-polypeptide moiety is a polymer molecule, enrichment in lysine residues is particularly desirable. Preferably, the diversified population of nucleotide sequences is enriched in codons specifying one or more amino acid residue(s) selected from the group consisting of Lys, Ser, Thr, Tyr, Cys, Asp and Glu, when the non-polypeptide moiety is a lipophilic compound. Preferably, the diversified population of nucleotide sequences encode polypeptides enriched in one or more amino acid residue(s) selected from the group consisting of an N-glycosylation site, arginine, histidine, cysteine, serine, threonine, hydroxyproline, phenylalanine, tyrosine and tryptophan, when the non-polypeptide moiety is a sugar moiety. Preferably, the diversified population of nucleotide sequences encode polypeptides enriched in one or more amino acid residue(s) selected from the group consisting of lysine, arginine, aspartic acid, glutamic acid, histidine and cysteine, when the non-polypeptide moiety is an organic derivatizing agent.

Focused mutagenesis is conveniently carried out by doping or spiking the mutagenic reaction with oligonucleotides. The doping or spiking can be designed on the basis of the skilled person's intelligent consideration of nucleotide coding parameters (in accordance with generally known principles), by use of a suitable algorithm, e.g. a computer program which is based on the algorithm described by Jensen et al. 1998 or Sedrovski and Mak (1993) (see above), or by using trinucleotides (Sondek, J. and Shortle, D., Proc. Natl. Acad. Sci, USA, Vol. 89, pp. 3581-3585, April 1992; Kayushin et al., Nucleic Acids Research, 1996, Vol. 24, No. 19, pp. 3748-3755; Virnekäs et al., Nucleic Acids Research, 1994, Vol. 22, No. 25; WO 93/21203).

In the present context, the term “enriched” is intended to indicate that the nucleotide sequence resulting from the mutagenesis contains more codons encoding the amino acid residue(s) in question than the unmutated nucleotide sequence or subsequence thereof. The term “enriched” is also intended to include the situation where one or more codons encoding the amino acid residue(s) in question is/are introduced into a sequence which does not contain such codons prior to mutagenesis.

In some circumstances, it is disadvantageous to have two or more attachment groups for the non-polypeptide moiety of choice located in close proximity to each other, because a heterogeneous population of polypeptide conjugates (such as polypeptide-polymer conjugates) can result if it is possible only to conjugate one of the two or more attachment groups (because of steric hindrance for conjugation of more than one group) or if only a subpopulation of the polypeptide conjugates have two or more attachments sites conjugated. More than one attachment site in a region can also increase the likelihood that an unnecessary decrease in function will occur. One way to avoid the introduction of more than one amino acid residue containing an attachment group into a given region is to conduct focused mutagenesis of this region in such a manner that each of the oligonucleotides employed in the focused mutagenesis encodes only one amino acid residue constituting an attachment group. This generally applicable approach is further described in Example 2.

In one embodiment, the diversified population of nucleotide sequences is designed so as to reduce the number of codons encoding an amino acid residue containing an attachment group, e.g., to remove two, three or four such amino acid residues from the polypeptide of interest. In particular, it is desirable to remove such amino acid residue(s) located at a functional site of the polypeptide in order to preserve or reduce a loss of function resulting from conjugation, e.g., glycosylation, PEGylation or other conjugation at such residue(s).

Also, if the polypeptide of interest contains two or more attachment groups located closely together (either in the primary or tertiary structure of the polypeptide), it can be advantageous to remove amino acid residues containing such groups in order to ensure that only one attachment group is available for conjugation within a given region of the polypeptide, thus, ensuring a more homogenous population of conjugated polypeptides. Accordingly, in a further embodiment, polypeptides having amino acid residues containing attachment groups that are separated by less than three residues in the primary sequence and/or having amino acids with attachment groups separated by less than 10 Å, preferably less than 8 Å, and more preferably less than 5 Å are targets for mutagenesis.

Preferably, the amino acid residues to be removed in the above embodiments are selected from the group consisting of lysine, arginine, aspartic acid, glutamic acid, tyrosine and cysteine, in particular lysine, when the non-polypeptide moiety is a polymer molecule; the group consisting of Lys, Ser, Thr, Tyr, Cys, Asp and Glu, when the non-polypeptide moiety is a lipophilic compound; the group consisting of an N-glycosylation site, arginine, histidine, cysteine, serine, threonine, hydroxyproline, phenylalanine, tyrosine and tryptophan, when the non-polypeptide moiety is a sugar moiety; and the group consisting of lysine, arginine, aspartic acid, glutamic acid, histidine and cysteine, when the non-polypeptide moiety is an organic derivatizing agent. The mutation should preferably be towards introduction of a residue which does not contain an attachment group, more preferably towards an amino acid residue present at the equivalent position in the protein sequence family in question and/or the towards a conservative amino acid substitution.

Examples of conservative substitutions are within the group of basic amino acids (such as arginine, lysine and histidine), acidic amino acids (such as glutamic acid and aspartic acid), polar amino acids (such as glutamine and asparagine), hydrophobic amino acids (such as leucine, isoleucine and valine), aromatic amino acids (such as phenylalanine, tryptophan and tyrosine), and small amino acids (such as glycine, alanine, serine, threonine and methionine). Amino acid substitutions that do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

Furthermore, doping or spiking to introduce or remove attachment groups can be designed so as to ensure a balanced number of attachment groups relative to the molecular weight and/or surface area of the polypeptide. For instance, the heavier the polypeptide is the more non-polypeptide moieties, such as polymer molecules, should be coupled to the polypeptide to obtain sufficient shielding of epitope(s) responsible for antibody formation.

When the non-polypeptide moiety to which the polypeptide of interest is to be conjugated is a sugar moiety, and the conjugation is conducted by way of in vivo glycosylation, the attachment group(s) to be introduced is a potential N-glycosylation site or O-glycosylation site. Accordingly, for this purpose the mutagenesis is conducted towards introduction of such N- or O-glycosylation site(s) at a suitable position in the polypeptide of interest. An N-glycosylation site can be introduced anywhere in the sequence by up to three mutations where the Asn residue is potentially exposed to the surface of the polypeptide of interest and is not located at the N-terminal residue. Preferably, localized or focused mutagenesis is performed to result in two or all of the residues being potentially exposed to the surface of the polypeptide of interest. Conveniently, the mutagenesis is conducted towards introduction of one, two or all of the amino acid residues making up the N-glycosylation site at positions where the equivalent position in another member of the protein sequence family has one or both of the mutation type residues (Asn or Ser/Thr), more preferably at positions where one of the residues is already present in the amino acid sequence of the polypeptide of interest (i.e., the Asn or the Ser or the Thr are in positions allowing a mutation of a conservative type, which in this particular context is defined as Asp->Asn, Gln->Asn, Ala->Ser, Gly->Ser, Ala->Thr, Gly->Thr.

### Conjugation

In a desirable embodiment of the method of the invention wherein the screening and selection steps are performed directly on the expressed polypeptides a) without a prior conjugation step, it can be desirable—after screening and selection—to conjugate selected polypeptides to a non-polypeptide moiety, e.g. to a polymer molecule, a lipophilic compound, a sugar moiety (e.g., by way of in vitro glycosylation) and/or an organic chemical derivative, in order to obtain a further decrease of immunogenicity and/or increase of functional in vivo half-life.

In other methods of the invention, conjugation to a non-polypeptide moiety is an integral step. It will be understood that such conjugation step only finds relevance when a non-polypeptide moiety other than an in vivo attached sugar moiety is to be conjugated to the polypeptide, since in vivo glycosylation takes place during the expression step when using an appropriate glycosylating host cell as expression host. Accordingly, whenever a conjugation step occurs in the present invention this is intended to be conjugation to a non-polypeptide moiety other than a sugar moiety attached by in vivo glycosylation. The polypeptide conjugate prepared by a method of the invention can comprise a variety of different numbers of non-polypeptide moieties, e.g. 1-20 non-polypeptide moieties, such as 1-10 or 2-10 non-polypeptide moieties.

In accordance with the invention conjugation to two or more different types of non-polypeptide moieties can be performed. For instance, the polypeptide expressed from the diversified population of nucleotide sequences can be conjugated to a polymer molecule and a lipophilic compound, to a polymer and a sugar moiety (e.g. by in vivo glycosylation), to a lipophilic compound and a sugar moiety (e.g. by in vivo glycosylation), etc. in order to obtain a further decrease of immunogenicity and/or increase of functional in vivo half-life. The conjugation to two or more different non-polypeptide moieties can be done simultaneously or sequentially. In the following sections “Conjugation to a lipophilic compound”, “Conjugation to a polymer molecule”, “Conjugation to a sugar moiety” and “Conjugation to an organic derivatizing agent” conjugation to specific types of non-polypeptide moieties is described.

Generally, it is desirable to conjugate various moieties, as described herein, to functional polypeptides. However, the methods of the invention are equally applicable to the diversification and selection of subportions of polypeptides that are useful biologically or experimentally in the absence of one or all of their native functions. For example, numerous immunogenic epitopes useful for the production of antibodies, e.g., as vaccines, or therapeutic or experimental reagents, require sugar attachments. The methods described herein can be employed to engineer epitopes that are favorably glycosylated in vivo or in vitro, regardless of whether the intact polypeptide retains function, or even whether the epitope resides within an larger polypeptide.

### Coupling to a Sugar Moiety

The coupling of a sugar moiety, or “glycosylation,” can take place in vivo or in vitro. Generally, glycosylation is classified as either “N-linked” or “O-linked” depending on the molecular nature of the attachment group. N- and O— linked glycosylation sites can be introduced according to the methods previously described herein, e.g., by doping or spiking with oligonucleotides corresponding to codons corresponding to N-linked and/or O-linked glycosylation sites. The terms “introduce” and “remove” as used in relation to a glycosylation site are primarily intended to mean substitution of amino acid residue(s), but may also mean insertion and deletion (without substitution), respectively. The introduction of an N-glycosylation site is conveniently achieved by introduction of one or more amino acid residues in the polypeptide in such a manner that a functional N-glycosylation site results. Analogously, an N-glycosylation site is removed by removal of one or more amino acid residues in the polypeptide in such a manner that an existing N-glycosylation site is destroyed.

In order to achieve in vivo coupling (i.e., in vivo glycosylation) of a polypeptide of interest which has been modified to introduce one or more glycosylation sites (see the section above entitled “Strategies for preparing a diversified population of nucleotide sequences”), the diversified population of nucleotide sequences must be inserted in a glycosylating, eucaryotic expression host. As a result of in vivo glycosylation attachment of sugar chains occurs in vivo, i.e., during posttranslational processing in a glycosylating cell used for expression of the polypeptide, e.g. by way of N-linked and/or O-linked glycosylation. The expression host cell can be selected from fungi-, insect- or animal cells, including human cells or from transgenic plant cells. In one embodiment the host cell is a mammalian cell, such as a Chinese hamster ovary (CHO) cell line, (e.g. CHO-K1; ATCC CCL-61), Green Monkey cell line (COS) (e.g. COS 1 (ATCC CRL-1650), COS 7 (ATCC CRL-1651)); mouse cell (e.g. NS/O), Baby Hamster Kidney (BHK) cell line (e.g. ATCC CRL-1632 or ATCC CCL-10), or human cell (e.g. HEK 293 (ATCC CRL-1573)), or any other suitable cell line, e.g., available from public depositories such as the American Type Culture Collection, Rockville, Md. Also, a mammalian glycosylation mutant cell line, such as CHO-LEC1, CHOL-LEC2 or CHO-LEC18 (CHO-LEC1: Stanley et al. Proc. Natl. Acad. USA 72, 3323-3327, 1975 and Grossmann et al., J. Biol. Chem. 270, 29378-29385, 1995, CHO-LEC18: Raju et al. J. Biol. Chem. 270, 30294-30302, 1995) may be used. Furthermore, an insect cell, such as a Lepidoptora cell line, e.g. Sf9, a plant cell line or a yeast cell, e.g. Saccharomyces cerevisiae, Pichia pastoris, Hansenula spp. can be used.

Covalent in vitro coupling of glycosides to amino acid residues of a polypeptide of interest can be used to modify or increase the number or profile of sugar substituents. The in vitro coupling is normally performed in a conjugation step of the present invention. Typically, in vitro glycosylation is a synthetic glycosylation reaction, performed in vitro, normally involving covalently linking a sugar chain to an attachment group of a polypeptide, optionally using a cross-linking agent. Depending on the coupling reaction used, the sugar(s) can be attached to a) arginine and histidine, b) free carboxyl groups, c) free sulfhydryl groups such as those of cysteine, d) free hydroxyl groups such as those of serine, threonine, tyrosine or hydroxyproline, e) aromatic residues such as those of phenylalanine or tryptophan or f) the amide group of glutamine. Suitable methods are described, for example in WO 87/05330 and in Aplin et al., CRC Crit Rev. Biochem., pp. 259-306, 1981.

In vitro glycosylation utilizes available attachment groups, e.g., that have been introduced according to the methods of the invention. Covalent in vitro coupling of oligosaccharide or glycoside based molecules (such as dextran) to amino acid residues of the polypeptide may be performed, e.g. as described, for example in WO 87/05330, by Aplin et al., CRC Crit Rev. Biochem., pp. 259-306, 1981, and by Doebber et al., J. Biol. Chem., 257, pp 2193-2199, 1982, the contents of which are incorporated herein by reference. For instance, Doebber et al. describe attachment of a synthetic Man3Lys2 glycopeptide to lysine residues by in vitro glycosylation. Furthermore, sugar moieties may be attached to the COOH group of an Asp, a Glu or the C-terminal amino acid residue of the polypeptide, to the SH group of a cysteine residue, to the aromatic group of a Phe, Tyr or Trp residue, To the guanidine group of an Arg residue, and to the imidazole ring of a His residue.

Furthermore, the in vitro coupling of sugar moieties or PEG to protein- and peptide-bound Gln-residues can be carried out by transglutaminases (TGases). Transglutaminases catalyse the transfer of donor amine-groups to protein- and peptide-bound Gln-residues in a so-called cross-linking reaction. The donor-amine groups can be protein- or peptide-bound e.g. as the ε-amino-group in Lys-residues or it can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al., Biochemistry 35, 1996, 13072-13080).

TGases, in general, are highly specific enzymes, and not every Gln-residues exposed on the surface of a protein is accessible to TGase-catalysed cross-linking to amino-containing substances. In order to render a protein susceptible to TGase-catalysed cross-linking reactions, stretches of amino acid sequence known to function well as TGase substrates are included, e.g., by oligonucleotide spiking, as described above. Several amino acid sequences are known to be or to contain excellent natural TGase substrates e.g. substance P, elafin, fibrinogen, fibronectin, α₂-plasmin inhibitor, α-caseins, and β-caseins and may thus be inserted into and thereby constitute part of the amino acid sequence of a polypeptide to be modified in accordance with the invention. Furthermore, the in vitro coupling of sugar moieties or PEG to protein- and peptide-bound Gln-residues of a polypeptide of interest can be carried out by transglutaminases (TGases). Transglutaminases catalyse the transfer of donor amine-groups to protein- and peptide-bound Gln-residues in a so-called cross-linking reaction. The donor-amine groups can be protein- or peptide-bound e.g., as the ε-amino-group in Lys-residues or it can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al. (1996) Biochemistry 35, 13072-13080).

If desired, the nature and number of sugar moieties (and thus determination of an altered glycosylation pattern) of a conjugated polypeptide prepared in accordance with the invention can be determined by a number of different methods known in the art e.g. by lectin binding studies (Reddy et al., 1985, Biochem. Med. 33: 200-210; Cummings, 1994, Meth. Enzymol. 230: 66-86; Protein Protocols (Walker ed.), 1998, chapter 9); by reagent array analysis method (RAAM) sequencing of released oligosaccharides (Edge et al., 1992, Proc. Natl. Acad. Sci. USA 89: 6338-6342; Prime et al., 1996, J. Chrom. A 720: 263-274); by RAAM sequencing of released oligosaccharides in combination with mass spectrometry (Klausen, et al., 1998, Molecular Biotechnology 9: 195-204); or by combining proteolytic degradation, glycopeptide purification by HPLC, exoglycosidase degradations and mass spectrometry (Krogh et al, 1997, Eur. J. Biochem. 244: 334-342).

### Conjugation to a Lipophilic Compound

The polypeptide and lipophilic compound are conjugated to each other, either directly or using a linker. The lipophilic compound can be a natural compound such as a saturated or unsaturated fatty acid, a fatty acid diketone, a terpene, a prostaglandin, a vitamin, a carotinoid or steroid, or a synthetic compound such as a carboxylic acid, an alcohol, an amine and sulphonic acid with one or more alkyl-, aryl-, alkenyl- or other multiple unsaturated compounds. The conjugation between the polypeptide and the lipophilic compound, optionally through a linker can be done according to methods known in the art, e.g., as described by Bodanszky in Peptide Synthesis, John Wiley, New York, 1976 and in WO 96/12505.

### Conjugation to a Polymer Molecule

The polymer molecule to be coupled to the polypeptide can be any suitable polymer molecule, such as a natural or synthetic homo-polymer or heteropolymer, typically with a molecular weight in the range of 300-100,000 Da, such as 300-20,000 Da, more preferably in the range of 500-10,000 Da, even more preferably in the range of 500-5000 Da.

Examples of homo-polymers include a polyol (i.e. poly-OH), a polyamine (i.e. poly-NH₂) and a polycarboxylic acid (i.e. poly-COOH). A hetero-polymer is a polymer, which comprises one or more different coupling groups, such as, e.g., a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, polyvinyl alcohol (PVA), poly-carboxylate, poly-(vinylpyrolidone), polyethylene-co-maleic acid anhydride, polystyrene-co-malic acid anhydride, dextran including carboxymethyl-dextran, or any other biopolymer suitable for altering immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms. PEG is the preferred polymer molecule to be used, since it has only few reactive groups capable of cross-linking compared, e.g., to polysaccharides such as dextran, and the like. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups. Suitably activated polymer molecules are commercially available, e.g. from Shearwater Polymers, Inc., Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which references are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide as well as the functional groups of the polymer (e.g. being amino, hydroxyl, carboxyl, aldehyde or sulfhydryl). The PEGylation can be directed towards conjugation to all available attachment groups on the polypeptide or a carbohydrate molecule linked thereto (i.e. such attachment groups that are exposed at the surface of the polypeptide) or can be directed towards specific attachment groups, e.g. the N-terminal amino group (U.S. Pat. No. 5,985,265). Furthermore, the conjugation can be achieved in one step or in a stepwise manner (e.g. as described in WO 99/55377).

Furthermore, the PEGylation step of a method of the invention can be designed so as to introduce a number of polymer molecules having a molecular weight, which number and weight are suitable for the polypeptide of interest and for achieving the desired effect of the PEGylation. For instance, if the primary purpose of the conjugation is to achieve a conjugate having a high molecule weight (e.g. to reduce renal clearance) it is usually desirable to conjugate as few high Mw polymer molecules as possible to obtain the desired molecular weight. When a substantial decrease of immunogenicity is desirable this can be obtained by use of a sufficiently high number of low molecular weight polymer (e.g. with a molecular weight of about 5,000 Da) to effectively shield all or most epitopes of the polypeptide. For instance, 2-8, such as 3-6 such polymers can be used.

In particular, extensive PEGylation can be employed when it is not critical to maintain a close to intact function of the polypeptide, since a normally observed drawback of too extensive PEGylation is that the function of the modified polypeptide is reduced. If a nearly intact function of the polypeptide of interest is desirable as well, the extensive PEGylation is conveniently performed according to the embodiment of the invention wherein a functional site of the polypeptide is blocked during PEGylation. If, on the other hand, it is critical to maintain a high function of the polypeptide of interest and less critical to obtain a substantially increased functional in vivo half-life and/or altered immunogenicity, the PEGylation should be designed so as to allow for a less extensive PEGylation.

In connection with conjugation to only a single attachment group on the protein (as described in U.S. Pat. No. 5,985,265), it can be advantageous that the polymer molecule, which can be linear or branched, has a high molecular weight, e.g. about 20 kDa.

Normally, the polymer conjugation is performed under conditions aiming at reacting all available polymer attachment groups with polymer molecules. Typically, the molar ratio of activated polymer molecules to polypeptide is 200-1, such as 100-1 and preferably 10-1 or 5-1 to obtain optimal reaction. However, also equimolar ratios of polypeptide to polymer may be used.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378.

Subsequent to the conjugation residual activated polymer molecules are blocked according to methods known in the art, e.g., by addition of primary amine to the reaction mixture.

### Coupling to an Organic Derivatizing Agent

Covalent modification of the polypeptide of interest can be performed by reacting (an) attachment group(s) of the polypeptide of interest with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonateat pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents can react with the groups of lysine as well as the arginine epsilon-amino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

### Blocking of Functional Site

It has been reported that excessive polymer conjugation often results in a loss of activity of the polypeptide in question. This problem can be eliminated, e.g., by removal of attachment groups located at the active site (according to one embodiment of the present invention discussed above in the section entitled “Strategies for creating a diversified population of nucleotide sequences) or by blocking the functional site prior to conjugation. This latter strategy constitutes a further embodiment of the invention. More specifically, in accordance with this embodiment the conjugation between the polypeptide and the non-polypeptide moiety is conducted under conditions where the functional site of the polypeptide is blocked by a helper molecule capable of binding to the functional site of the polypeptide. Preferably, the helper molecule is one, which specifically recognizes the functional site of the polypeptide. The helper molecule can, e.g., be a low molecular weight ligand, a receptor or the like.

The polypeptide is allowed to interact with the helper molecule before effecting conjugation. This ensures that the functional site of the polypeptide is shielded or protected and consequently unavailable for derivatization by the non-polypeptide moiety such, as a polymer. Following its elution from the helper molecule, the conjugate between the non-polypeptide moiety and the polypeptide can be recovered with at least a partially preserved functional site.

For instance, when the polypeptide of interest is an antibody the helper molecule can be, e.g., an antigen or an anti-idiotypic antibody. When the polypeptide of interest is a cytokine the helper molecule can be a receptor or a specific antibody. When the polypeptide of interest is an antigen the helper molecule can be an antibody. When the polypeptide of interest is an enzyme the helper molecule can be an enzyme inhibitor or an antibody. When the polypeptide of interest is a ligand the helper molecule can be a receptor or antibody. When the polypeptide of interest is a receptor the helper molecule can be a ligand or antibody. Specific examples of pairs of polypeptide of interest and helper molecule include the following:

Streptokinase or staphylokinase—plasminogen; hirudin—thrombin; a hormone—the specific receptor; a growth factor—a growth factor receptor; a cytokine—the corresponding cytokine receptor; a fibrinolytic enzyme such as pro-urokinase, urokinase or tPA—benzamidine or a derivative thereof; a heparin-binding protein such as a growth factor—heparin, a heparin-like molecule or a heparin derivative, in particular one with a low molecular weight and a negative charge; a DNA binding protein—DNA or an oligonucleotide.

In some instances it can be desirable to preserve the biological activities mediated by two or more separate functional sites of the polypeptide of interest. In such cases both biological activities can be preserved through the use of two or more specific binders each recognizing one of the two or more functional sites.

The subsequent conjugation of the polypeptide having a blocked functional site to a polymer, a lipophilic compound, a sugar moiety, an organic derivatizing agent or any other compound is conducted in the normal way, e.g., as described in the sections above entitled “Conjugation to . . . ”.

Irrespectively of the nature of the helper molecule to be used to shield the functional site of the polypeptide of interest from conjugation, it is desirable that the helper molecule is free from or comprises only a few attachment groups for the non-polypeptide moiety of choice in part(s) of the molecule, where the conjugation to such groups will hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free from conjugatable epsilon amino groups, preferably free from any epsilon amino groups. Accordingly, in a preferred embodiment the helper molecule is a protein or peptide capable of binding to the functional site of the polypeptide, which protein or peptide is free from any conjugatable attachment groups for the non-polypeptide moiety of choice.

Of particular interest in connection with the embodiment of the present invention wherein the polypeptide conjugates are prepared from a diversified population of nucleotide sequences encoding a polypeptide of interest, the blocking of the functional group is effected in microtiter plates prior to conjugation, for instance, by plating the expressed polypeptide variant in a microtiter plate containing an immobilized blocking group such as a receptor, an antibody or the like.

In a further embodiment the helper molecule is first covalently linked to a solid phase such as column packing materials, for instance Sephadex or agarose beads, or a surface, e.g. reaction vessel. Subsequently, the polypeptide is loaded onto the column material carrying the helper molecule and conjugation carried out according to methods known in the art, e.g. as described in the sections above entitled “Conjugation to . . . ”. This procedure allows the polypeptide conjugate to be separated from the helper molecule by elution. The polypeptide conjugate is eluated by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule can be derivatised with a second molecule (e.g. biotin) that can be recognized by a specific binder (e.g. streptavidin). The specific binder can be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate can be released from the helper molecule in any appropriate fashion. Deprotection can be achieved by providing conditions in which the helper molecule dissociates from the functional site of the polypeptide of interest to which it is bound. For instance, a complex between an antibody to which a polymer is conjugated and an anti-idiotypic antibody can be dissociated by adjusting the pH to an acid or alkaline pH.

### Conjugation of a Tagged Polypeptide

In an alternative embodiment the polypeptide of interest is expressed, as a fusion protein, with a tag, i.e., an amino acid sequence or peptide stretch made up of typically 1-30, such as 1-20 amino acid residues. Besides allowing for fast and easy purification, the tag is a convenient tool for achieving conjugation between the tagged polypeptide of interest and the non-polypeptide moiety. In particular, the tag can be used for achieving conjugation in microtiter plates or other carriers, such as paramagnetic beads, to which the tagged polypeptide can be immobilised via the tag. The conjugation to the tagged polypeptide of interest in, e.g., microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag can function as a spacer molecule ensuring an improved accessibility to the immobilised polypeptide to be conjugated. The conjugation using a tagged polypeptide can be to any of the non-polypeptide moieties disclosed herein, e.g. to a polymer molecule such as PEG. The identity of the specific tag to be used is not critical as long as the tag is capable of being expressed with the polypeptide and is capable of being immobilised on a suitable surface or carrier material. A number of suitable tags are commercially available, e.g. from Unizyme Laboratories, Denmark. For instance, the tag can any of the following sequences:

His-His-His-His-His-His
Met-Lys-His-His-His-His-His-His
Met-Lys-His-His-Ala-His-His-Gln-His-His
Met-Lys-His-Gln-His-Gln-His-Gln-His-Gln-His-Gln-
His-Gln

(all available from Unizyme Laboratories, Denmark)
or any of the following:

EQKLI SEEDL
(a C-terminal tag described in Mol. Cell. Biol.
5:3610-16, 1985)
DYKDDDDK
(a C- or N-terminal tag)
YPYDVPDYA

Antibodies against the above tags are commercially available, e.g. from ADI, Aves Lab and Research Diagnostics.

A convenient method for using a tagged polypeptide for PEGylation is given in the Materials and Methods section below.

The subsequent cleavage of the tag from the polypeptide can be achieved by use of commercially available enzymes.

### Method of Avoiding Conjugation of the N-Terminal Amino Acid Residue

In a further aspect the invention relates to a generally applicable method for avoiding conjugation at the N-terminal amino acid residue of a polypeptide of interest, e.g. any of those mentioned in the section entitled “Polypeptide of interest” and preferably a therapeutically active polypeptide such as a cytokine or a hormone or an industrially useful polypeptide such as an enzyme. More specifically, this aspect relates to a method of preparing a polypeptide conjugate comprising a polypeptide of interest and a non-polypeptide moiety, the non-polypeptide moiety being able to attach to the N-terminal amino acid residue of the polypeptide and the polypeptide having an N-terminal Gln residue, which method comprises derivatizing the N-terminal Gln residue with glutamine cyclotransferase to obtain a pyro Glu residue, and subjecting the resulting derivatized polypeptide to conjugation. The method according to this aspect finds particular interest where conjugation to an N-terminal amino acid residue is undesirable. The pyro Glu residue cannot be conjugated and thus N-terminal conjugation is avoided.

The non-polypeptide moiety can be any of those described in the section entitled “Conjugation to a polymer molecule”, and is preferably polyethylene glycol (PEG). The conjugation can be achieved as described in that section.

The derivatization of the N-terminal Gln is done with glutamine cyclotransferase in accordance with established techniques, e.g. as recommended by the manufacturer. The enzyme can be purchased from Unizyme, Denmark.

Normally, the N-terminal Gln residue has been introduced into the polypeptide sequence, either by substitution of the N-terminal residue of the parent polypeptide, or by addition of the Gln residue to said N-terminal residue. The substitution or addition can be accomplished by methods known in the art.

The method according to this aspect normally further comprises removing the pyro Glu residue, conveniently by use of pyroglutamyl aminopeptidase in accordance with established techniques. The enzyme is, e.g., available from Unizyme, Denmark. Removal of the pyro Glu residue is relevant, when the presence of such residue impairs the function of the conjugate. Furthermore, the presence of such residue in conjugates intended for therapeutic use is normally undesirable.

### Screening

It will be understood that the screening for improved properties, such as function, immunogenicity and/or functional in vivo half-life is designed on the basis of the desired result to achieve. If, for instance, a method of the invention is used to alter the immunogenicity of an otherwise functional polypeptide of interest the screening step will primarily be designed so as to screen for altered, i.e., reduced or increased immunogenicity, whereas no screening for function or functional in vivo half-life is conducted. Typically, it is desirable to employ high throughput screening methods in conjunction with the methods of the invention. High throughput is typically in excess of 100, frequently in excess of 1000, and often in excess of 10,000 samples per day. Numerous formats for accomplishing high throughput screening are known in the art. Among the more common formats are microtiter plates, pin arrays, bead arrays, membranes, filters and microfluidic devices.

One standard format for the performance of high throughput assays is microtiter plates. Microtiter plates with 96, 384 or 1536 wells are widely available, however other numbers of wells, e.g., 3456 and 9600 are also used. In general, the choice of microtiter plates is determined by the handling and/or analytical device to be used, e.g., automated loading and robotic handling systems. Exemplary systems include the ORCA™ system from Beckman-Coulter, Inc. (Fullerton, Calif.) and the Zymate systems from Zymark Corporation (Hopkinton, Mass.).

Alternatively, other formats such as “chip” or pin arrays, or formats involving immobilization of one or more assay component on a solid support such as a membrane or filter, e.g., nylon, nitrocellulose, and the like, are employed in high throughput assays useful in the context of the present invention. In addition, numerous assays useful in detecting proteins, or cells expressing proteins, with desirable properties can be performed in microfluidic devices such as the LabMicrofluidic Device™ high throughput screening system (HTS) by Caliper Technologies Corp., Mountain View, Calif., or the HP/Agilent technologies Bioanalyzer using LabChip™ technology by Caliper Technologies Corp. See, also, www.calipertech.com.

### Screening for Function

As indicated above the function/functions for which screening is to be performed depend on the nature of the polypeptide of interest. Typically, the function to be screened for is selected from the group consisting of activity, affinity, potency, efficiency, specificity and selectivity. For instance, when the polypeptide is an enzyme, the function to be screened for will typically be selected from the group consisting of enzymatic activity, substrate specificity, substrate affinity, temperature optimum, pH optimum, thermostability, pH tolerance, tolerance towards components with which the enzyme is in contact under its normal use, enzyme kinetic parameters such as Vmax or Km, etc. When the polypeptide of interest is an antibody, the function to be screened for is typically the antibody's ability to bind or the affinity for an antigen or epitope. When the polypeptide of interest is a hormone or an interleukin the function to be screened for is typically the receptor affinity, receptor signalling capability, activity, specificity, potency or selectivity. When the polypeptide of interest is a regulatory protein the function to be screened for is typically affinity, specificity or selectivity. The screening can be conducted according to principles well known in the art for screening for the function in question.

Conveniently, the screening for function is conducted in microtiter plates, in particular in the plates containing the polypeptide conjugate resulting from the conjugation step of a method of the invention. Preferably, the screening is a high throughput screening. In the context of the present application “microtiter plates” are to be understood broadly to comprise not only microtiter plates in its conventional meaning, but also chips and other solid phases suitable for screening a high number of samples in a short time, as described above. In accordance with the specific embodiment of the invention wherein a functional site of the polypeptide of interest is blocked during the polypeptide conjugation step, the screening for function can be omitted in that only functional polypeptides capable of binding to the blocking group will be conjugated to the non-polypeptide moiety, such as a polymer.

### Screening for Altered Immunogenicity

Conveniently, the screening for altered immunogenicity is performed by contacting the polypeptide conjugate with an antibody recognizing the non-conjugated polypeptide and detecting the amount of antibody reacting with the conjugate. The detection of the amount of antibody is done in accordance with standard immunochemistry methods known in the art. For instance, the detection method is based on the use of secondary antibody, such as an anti-human antibody, conjugated to an enzyme catalyzing a measurable reaction with subsequent detection of the enzyme activity. The enzyme can, e.g., be horseradish peroxidase. The detection method can also be based on a method wherein the antibody and/or the secondary antibody is/are labeled with a fluorescent probe. Furthermore, the secondary antibody can be labeled with a radioactive probe such as I-125 or H-3. The screening for altered immunogenicity is conveniently performed in microtiter plates.

### Screening for Function and Altered Immunogenicity

In a highly preferred embodiment the screening for function and altered, in particular reduced immunogenicity is performed simultaneously. More specifically, the screening can be conducted in parallel, i.e. subjecting the population of polypeptide conjugates resulting from the conjugation step of a method of the invention to parallel screening for function and immunogenicity, respectively, and selecting polypeptide conjugates which have altered immunogenicity and a measurably function relative to the polypeptide of interest. Alternatively, the screening for function and altered immunogenicity can be performed as one screening, when a functional site of the polypeptide of interest is blocked as described in the section entitled “Blocking of a functional site” (thereby inherently resulting in a functional polypeptide) and the screening to be conducted is for altered immunogenicity as described above.

Preferably, the simultaneous screening for function and altered immunogenicity is done in microtiterplates. An advantage of using microtiter plates is that the screening can be performed as a high throughput screening. In a highly preferred embodiment of a method of the present invention the polymer conjugation and the screening are performed in the same microtiter plates. This ensures an efficient high throughput screening procedure.

### Secondary Screening

In addition to or as an alternative to the above primary screening procedures a secondary screening for function or immunogenicity is normally performed.

A secondary screening for function is conveniently conducted by isolating the polypeptide conjugate and subjecting the isolated conjugate to a suitable test for the function in question. Relevant functions for different types of polypeptides of interest are exemplified in the section above termed “Screening for function”. The secondary screening can be conducted in accordance with methods known in the art for assessing the function in question.

A secondary screening for altered immunogenicity is conveniently conducted by injecting an animal subcutaneously with the modified polypeptide or polypeptide conjugate and comparing the response with the response of the corresponding unmodified or non-conjugated polypeptide of interest. A number of in vitro animal models exist for assessment of the immunogenic potential of polypeptides. Some of these models give a suitable basis for hazard assessment in man. Suitable models include mice, rabbit and hamster model. One model seeks to identify the immune response in the form of the IgG response in Balb/C mice being injected subcutaneously with a modified polypeptide or polypeptide conjugate and the unmodified or non-conjugated polypeptide of interest, respectively. For Balb/C mice the IgG response gives a good indication of the immunogenic potential of polypeptides. Also other animal models can be used for assessment of the immunogenic potential.

A polypeptide having “altered immunogenicity” according to the invention gives rise to a decreased or increased immune reaction, e.g., reflected in reduced or increased amount of produced antibodies in comparison to the polypeptide of interest.

### Screening for Increased Functional In Vivo Half-Life

The screening for increased functional in vivo half-life can be conducted in accordance with methods known in the art for assessing functional in vivo half-life. For example, BALB/c mice are injected intravenously, intramuscularly or subcutaneously with a suitable amount of the modified polypeptide to be analysed and blood samples collected at suitable time intervals in order to be able to determine the functional in vivo half-life of the polypeptide. Examples of suitable methods are described by He et al., Life Sciences, Vol. 64, No. 14, pp. 1163-1175, 1999 and Pettit et al., the Journal of Biological Chemistry, Vol. 272, No. 4, p. 2312-2318, 1997.

### Analysis of Polypeptide Conjugates Selected in a Method of the Invention

Once a suitable modified polypeptide, in particular a polypeptide conjugate, constructed according to the invention has been selected in a screening step of a method of the invention the nucleotide sequence encoding the polypeptide part of the conjugate is isolated and used for expression of larger amounts of the polypeptide (see below). The amino acid sequence of the resulting polypeptide is determined and the polypeptide is subjected to conjugation in a larger scale. Subsequently, the polypeptide conjugate is assayed with respect to immunogenicity and/or functional in vivo half-life. The polypeptide part of the conjugate or the polypeptide resulting from the method according to the first aspect of the invention is termed “modified polypeptide”.

Preparing a Polypeptide Conjugate Resulting from a Method of the Invention

Once the modified polypeptide, in particular the polypeptide conjugate, has been analysed it can be produced in a larger scale, such as for commercial purposes, using methods known in the art.

The modified polypeptide is conveniently produced by recombinant expression technology known in the art. In brief, a nucleotide sequence encoding the polypeptide is inserted into a suitable expression vector with which a suitable host cell is subsequently transformed or transfected. Alternatively, the nucleotide sequence is directly inserted into the host cell. In the host cell the nucleotide sequence encoding the polypeptide is operably linked to all the control sequences required for expression of the sequence. The nucleotide sequence can be single- or double-stranded and can include, but is not limited to, DNA, cDNA, and recombinant nucleic acid sequences.

The term “control sequences” is defined herein to include all components which are necessary or advantageous for the expression of the modified polypeptide. Each control sequence can be native or foreign to the nucleic acid sequence encoding the polypeptide. Such control sequences include, but are not limited to, a leader, polyadenylation sequence, propeptide sequence, promoter, enhancer or upstream activating sequence, signal peptide sequence, and transcription terminator. At a minimum, the control sequences include a promoter, and transcriptional and translational stop signals. The choice of host cell will depend, to a large extent, on the nature of the polypeptide to be produced, including its origin, the quantity of polypeptide required, and the intended use of the polypeptide. Furthermore, any need for posttranslational modification by the host will influence the choice of host. Examples of host cells that can be used include mammalian, insect or microbial cells, such as bacterial, yeast or fungal cells. Suitable expression systems which are generally known by the skilled person include a mammalian expression system based on CHO, BHK or COS cells (see in, vivo glycosylation section above), an insect cell expression system such as SF9 cells, a yeast expression system based on Saccharomyces cerevisiae, Pichia such as P. pastoris or P. methanolica or Hansenula, such as H. polymorpha, a bacterial expression system based on Bacillus, such as B. subtilis, or Eschericiae coli, and a fungal expression system based on Aspergillus, Fusarium or Trichoderma. Transformation of any of these cells with the nucleotide sequence encoding the modified polypeptide is performed in accordance with well-known methods for such transformation.

The recombinant production of the polypeptide is normally achieved by cultivating the resulting host cell containing a nucleotide sequence encoding the modified polypeptide under conditions conducive for the production of the polypeptide, and recovering the polypeptide. If the polypeptide is produced as an extracellular product it is normally recovered directly from the medium. If it is produced as an intracellular polypeptide it is normally recovered after disrupture of the cells resulting from the cultivation. Subsequent to being recovered the polypeptide can be subjected to further purification or other treatment.

Subsequent to recovery and possible other treatment, the polypeptide is subjected to conjugation to the non-polypeptide moiety according to methods known in the art. The conjugation is carried out under conditions ensuring the same degree and nature of conjugation as that found in the polypeptide molecule being selected in a method of the invention.

In the present application reference has been made to a number of publications, the contents of which should be considered to be incorporated herein by reference. In the following non-limiting examples methods of the present invention are exemplified using staphylokinase as an illustrative example of a polypeptide of interest. The examples should not, in any manner, be construed as limiting the generality of the present invention.

### EXAMPLES

### Materials

Plasmin substrate S-2251/H-D-Val-Leu-Lys-pNA from Chromogenix

pET12a expression vector (Novagen, Inc., Studier et al. Methods of Enzymology 185, 60-89, 1990).

The E. coli strains BL21(DE3), B834(DE3), AD494(DE3) or BLR(DE3) (Novagen, Inc., Studier et al. Methods of Enzymology 185, 60-89, 1990)

### Media

### LB Medium:

### Per Liter:

10 g Bacto tryptone

5 g yeast extract

10 g NaCl

Adjust pH to 7.5 and autoclave.

### Methods

### Construction of a Protein Sequence Family

The construction of a protein sequence family from a single protein amino acid sequence can be performed in a number of ways. For instance, the sequence family can be provided from a publicly available pre-constructed protein sequence family, e.g. the PFAM families database (http://pfam.wustl.edu/) (Nucleic Acids Res 1999 Jan. 1; 27(1):260-2) version 4.0 or the PROSITE data base Hofmann K., Bucher P., Falquet L., Bairoch A. The PROSITE database, its status in 1999 Nucleic Acids Res. 27:215-219 (1999). Furthermore, the protein sequence family can be provided from recursive searches in protein sequence databases like SWISS-PROT or TrEMBL Bairoch A., Apweiler R. The SWISS-PROT protein sequence data bank and its supplement TrEMBL in 1999 Nucleic Acids Res. 27:49-54 (1999) using well established sequence search/comparison algorithms like FASTA (Pearson W. R. and Lipman D. J. (1981) Proc. Natl. Acad. Sci. U.S.A. 85. 2444-2448), BLAST (Altshul, S. F. et. al. (1997) Nucleic Acids Res. 25. 3389-3402), PSI-BLAST (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) or from searches in nucleotide sequence data bases like EMBL (Guenter Stoesser*, Mary Ann Tuli, Rodrigo Lopez and Peter Sterk, Nucleic Acids Research, 1999, 27(1):18-24) or GENEBANK (Benson D A, Boguski M S, Lipman D J, Ostell J, Ouellette B F, Rapp B A, Wheeler D L. Nucleic Acids Res 1999, 27(1):12-17) using equally well established search algorithms. An overview of these methods can be found in Trends Guide to Bioinformatics (1998) Elsevier Science. The sequences of the members of the protein sequence family can be aligned using standard software, e.g. CLUSTALW, version 1.74 (Thompson, J. D., Higgins, D. G. and Gibson, T. J. (1994) CLUSTAL W: improving the sensitivity of progressive multiple sequence alignment through sequence weighting, positions-specific gap penalties and weight matrix choice, Nucleic Acids Research, 22:4673-4680).

### Building a Model Structure

A model structure can easily be constructed by the skilled person on the basis of the known three-dimensional structure of another member of the polypeptide sequence family to which the polypeptide of interest belongs. In order to be able to construct a model structure it is normally desirable that the polypeptide of interest displays at least 30% sequence identity with the polypeptide with the known three-dimensional structure. The model structure can be constructed using any suitable software known in the art, such as, for example, the software Modeller (Andrej Sali, Roberto Sánchez, Azat Badretdinov, András Fiser, and Eric Feyfant, The Rockefeller University, 1230 York Avenue, New York, N.Y. 10021-6399, USA) or the software WHAT IF: A molecular and drug design program (G. Vriend, J. Mol. Graph. (1990) 8, 52-56).

### Methods for Use in Determining Target Locii for Modification of a Polypeptide of Interest

### A: A: Analysis of the Polypeptide Structure or Sequence

### 1. Accessible Surface Area:

From a three-dimensional structure of the polypeptide of interest (e.g. X-ray, NMR or model structure) the surface accessibility of the individual atoms can be computed using state of the art software, e.g. NACCESS (c) S. Hubbard and J. Thornton 1992-6, http://sjh.bi.umist.ac.uk/naccess.html, What If (se reference above) or similar software, e.g. Biosym/Insight II. These methods typically use a probe-size of 1.4 Å and define the Accessible Surface Area (ASA) as the area formed by the center of the probe. Prior to this calculation all water molecules, all hydrogen atoms and other atoms not directly related to the protein (such as unrelated metal ions, co-factors and the like) are removed from the coordinate set.

### 2. Determine Residues Potentially Exposed to the Surface:

In order to determine residues potentially exposed to the surface of the polypeptide of interest the following steps are performed:

If a structure or a model structure is available residues are considered to be exposed to the surface if any part of any side chain atom (i.e. excluding the backbone atoms N, C, CA, and O for all amino acid residues except Gly) is in contact with the solvent. If the residue is a Glycine (Gly) the residue is considered to be exposed to the surface if the CA atom has any contact with the solvent. In this respect contact with the solvent is defined as any non-zero accessible surface area (ASA).

If a protein sequence family comprising the polypeptide of interest is available, any residue equivalent to a hydrophilic residue (Asp, Glu, His, Lys, Asn, Gln, Arg, Ser, Thr, Tyr) or Gly in any of the other members of the protein sequence family, the amino acid sequence of which has at least 40% identity to the amino acid sequence of the polypeptide of interest, is regarded as potentially exposed to the surface.

If a structure, a model structure or a protein sequence family is not available any hydrophilic residue (see above) and Gly are regarded as potentially exposed to the surface.

In the case where a structure of the complete sequence is not available but a structure of a part of the sequence is available a combination of a), b) and c) is applied, i.e. a) is used for the part where a structure is available and b) and/or c) is used for the remaining part.

### B: Selection of Amino Residues or Regions to be Mutagenized

The method for selection of residues or regions to be mutagenized comprises the following steps:

1: Construct a protein sequence family on the basis of the amino acid sequence of the polypeptide of interest

2: Determine residues potentially exposed to the surface (see section A above)

3: If a three-dimensional structure is available:

a) Determine the distance from the CB in each residue (in the case of Gly from the CA) to CB of all amino acid residues containing an attachment group, such as a polymer attachment group.

Determine the distance from the CB in each residue (in the case of Gly from the CA) to the (polymer) attachment group of all residues of the type to be modified (NZ in the case of a Lys, CG in the case of an Asp, CE in the case of a Glu, N for the N-terminal residue and the C of the C-terminal residue, etc.).

c) Determine the shortest distance from the (polymer) attachment group of each amino acid residue containing such group to the (polymer) attachment group of any other amino acid residue containing such group.

4: Determine the residues located at a functional site.

5: Determine Epitopes. Determination of epitopes can be performed using any conventional method known in the art, e.g. by use of epitope mapping as described by Ivan Roitt in Essential Immunology (Blackwell Scientific Publications, 1994, in particular pp. 118-120 thereof) and in the Detailed Description of the Invention section herein.

### Epitope Mapping

Several techniques exist for identification of epitopes on a polypeptide of interest (i.e. epitope mapping), see, e.g. Romagnoli et al., Biol Chem, 1999, 380(5):553-9, DeLisser H M, Methods Mol Biol, 1999, 96:11-20, Van de Water et al., Clin Immunol Immunopathol, 1997, 85(3):229-35, Saint-Remy J M, Toxicology, 1997, 119(1):77-81, and Lane D P and Stephen C W, Curr Opin Immunol, 1993, 5(2):268-71. One method is to establish a phage display library expressing random oligopeptides of e.g. 9 amino acid residues. IgG1 antibodies from specific antisera towards the polypeptide of interest are purified by immunoprecipitation and the reactive phages are identified by immunoblotting. By sequencing the DNA of the purified reactive phages, the sequence of the oligopeptide can be determined followed by localization of the sequence on the 3D-structure of the polypeptide of interest. The thereby identified region on the structure constitutes an epitope, which then can be selected as a target region for introduction of an attachment group for a non-polypeptide moiety or for destruction of the epitope.

### Assay for Staphylokinase Based on a Chromogenic Assay

The principle behind a chromogenic assay is to measure enzymatic activity through an enzyme-dependent liberation of a chromophore from a precursor acting as a substrate for the enzyme (a chromogenic substrate). As complexes between staphylokinase and plasmin act as plasminogen-activators the basis for the chromogenic assay for staphylokinase activity is that catalytic amounts of staphylokinase-plasmin complexes activate plasminogen to plasmin.

In the investigation of plasminogen-activating efficacy of different staphylokinase variants, differences in the kinetic behavior of chromophore liberation in this assay will reflect differences in the plasminogen-activator activity of different staphylokinase-plasmin complexes and thus of the different staphylokinase variants tested.

To conduct the assay, staphylokinase or staphylokinase conjugate is incubated with a small molar excess of plasmin at 37° C. Following the complex-formation period, catalytic amounts of the staphylokinase-plasmin complex is added to plasmin-free plasminogen at 37° C. At well-defined time points aliquots are withdrawn and added to a commercially available chromogenic substrate, e.g. S-2251/H-D-Val-Leu-Lys-pNA for plasmin, and allowed to react for a specified period of time at 37° C. Finally, the colour developed is measured using spectrophotometry. Evaluation of the activation kinetics is carried out by comparison to the colour development found for the relevant reagent and reaction controls.

In this way the formation of plasmin from plasminogen caused by staphylokinase and staphylokinase-polymer conjugate can be followed kinetically. Based upon this it is possible to evaluate the plasminogen-activating efficacy of staphylokinase and conjugates thereof and thus to assess whether or not the staphylokinase conjugate has a measurable function.

Detection of Antibody Interaction with Conjugated Polypeptide

Immobilized, conjugated polypeptide, such as conjugated staphylokinase, is incubated with antibodies known to react with the non-conjugated polypeptide. The amount of bound antibody is determined using a standard ELISA assay. More specifically, the assay is performed in microtiter plates in which the conjugated polypeptide is covalently attached. The primary antibodies employed can be human antibodies from patients previously exposed to the non-conjugated polypeptide of interest, or polyclonal or monoclonal antibodies raised in animals (e.g. goat, rabbit, mouse, donkey) using the polypeptide of interest in its entirety or peptides sequences from it. Appropriate secondary antibodies conjugated to alkaline phosphatase are used for the detection of the amount of primary antibody bound. The assay is conducted according to the following procedure: Wash coated plate 3× with Wash Buffer (1×PBS (0.058 M Na₂HPO₄, 0.017 M NaH₂PO₄, 0.068 M NaCl), 0.1% Tween-20).

Block nonspecific binding by incubating wells with Blocking Buffer (0.5% BSA or skimmed milk powder, 0.05% Tween-20 in PBS (0.058 M Na₂HPO₄, 0.017 M NaH₂PO₄, 0.068 M NaCl).

Wash wells 3× with Wash Buffer.
Dilute primary antibody and test antigen samples in Blocking Buffer and add 100 μl/well.
Incubate for at least 1 hour at room temperature with shaking.
Wash wells 3× with Wash Buffer.
Dilute secondary antibody-alkaline phosphatase conjugate in Blocking Buffer and add 100 μl/well. Incubate for at least 1 hour with shaking.
Wash wells 4× with Wash Buffer.
Add standard detectable alkaline phosphatase substrate; incubate for 5-10 min; and measure chemiluminescence at 5 min intervals.

### PEGylation in Microtiter Plates of a Tagged Polypeptide of Interest

The method comprises

expressing the polypeptide of interest with a suitable tag, e.g. any of the tags exemplified in the general description above.

Transferring culture broth to one or more wells in a microtiter plate capable of immobilising the tagged polypeptide. When the tag is His-His-His-His-His-His (Casey et al, J. Immunol. Meth., 179, 105 (1995)), a Ni-NTA HisSorb microtiter plate commercially available from QiaGen can be used.

After allowing for immobilising the tagged polypeptide to the microtiter plate, the wells are washed in a buffer suitable for binding and subsequent PEGylation. Incubating the wells with the activated PEG of choice. As an example, M-SPA-5000 from Shearwater Polymers is used. The molar ratio of activated PEG to polypeptide has to be optimised, but will typically be greater than 10:1 more typically greater than 100:1. After a suitable reaction time at ambient temperature, typically around 1 hour, the reaction is stopped by removal of the activated PEG solution. The conjugated protein is eluted from the plate by incubation with a suitable buffer. Suitable elution buffers can contain Imidazole, excess NTA or another chelating compound.

The conjugated protein is assayed for biological activity and immunogenicity as appropriate.

This tag can optionally be cleaved off using a method known in the art, e.g. using diaminopeptidase and the Gln in pos −1 will be converted to pyroglutamyl with GCT (glutamylcyclotransferase) and finally cleaved off with PGAP (pyro-glutamyl-aminopeptidase) giving the native protein. The process involves several steps of metal chelate affinity chromatography. Alternatively, the tagged polypeptide can be conjugated.

### Example 1

### Cloning and Expression of the Staphylokinase Gene

### Staphylokinase

Staphylokinase is a single chained polypeptide consisting of 136 amino acid residues without disulfide-bonds and cysteine-residues. The three dimensional structure of staphylokinase has been determined both by x-ray crystallography and by NMR showing that the protein is folded into a single domain (Rabijns et al., Nat. Struct. Biol. 4: 357 (1997); Ohlenschlager et al., Biochemistry: 37 (1998)). In addition, the three dimensional structure of staphylokinase in complex with μ-plasmin has been determined (Parry et al., Nat. Struct. Biol. 5: 917 (1998)).

The B cell epitopes of staphylokinase have recently been mapped using a phage-displayed library of staphylokinase variants selected for mutants that escaped binding to an affinity matrix derivatised with patient-specific polyclonal anti-staphylokinase antibodies (Jenné et al., J. Immunol. 161: 3161 (1998)). The main B cell epitopes were primarily found in two large discontinous areas covering 35% of the solvent-accessible surface of staphylokinase.

### Cloning and Expression of the Staphylokinase Gene

A synthetic gene is constructed on the basis of the so called SakSTAR gene encoding a Staphylococcus aureus staphylokinase variant (SEQ ID NO 3), which as compared to the wildtype S. aureus staphylokinase (SEQ ID NO 2) contains the mutation G34S and which codes for a protein having superior thermostability properties (Gase et al., Eur. J. Biochem, 223, 303-308 (1994)). The synthetic gene is constructed with a SalI site at the 5′-end just before the first amino acid codon of the mature staphylokinase and a BamHI at the 3′-end just after the termination codon. The SalI site is designed so the staphylokinase sequence is in frame with the ompT leader sequence of the pET12a expression vector. The synthetic gene is cloned into the SalI site and BamHI site of pET12a, which carries an N-terminal ompT signal sequence for periplasmic export of the staphylokinase. The E. coli strains BL21(DE3), B834(DE3), AD494(DE3) or BLR(DE3) are transformed with the resulting vector.

For expression of the staphylokinase, a single colony from one of the transformants is inoculated into LB medium with 50 ug/l ampicillin and grown overnight at 37° C. 2 ml are used to inoculate 50 ml LB medium with 50 ug/l ampicillin and grown with shaking until OD_{600 nm} reaches 0.4 to 1.0. Then IPTG is added to a final concentration of 0.4 mM and the incubation is continued for 5 hours at 30° C. The flasks are placed on ice and the cells pelleted by centrifugation. The staphylokinase is purified from the cell supernatant or from the periplasmic fraction of the cells. The periplasmic fraction is prepared by osmotic shock of the cells.

For large scale preparations the volumes are scaled up.

### Example 2

### Preparing a Diversified Population of Nucleotide Sequences Encoding Staphylokinase Modified to Increase the Number of Lysine Residues in a Target Locus of Choice

The sequence of Staphylococcus aureus Staphylokinase is available via SwissProt entry SAK_STAAU accession number P00802. The sequence of the mature protein consists of 136 residues and is shown in SEQ ID NO 2.

The three-dimensional X-ray crystallography structure of the C-terminal part (constituting amino acid residues Ser16 to Lys136) of the G34S mutant of the S. aureus staphylokinase, which has the amino acid sequence SEQ ID NO 3 and which has superior thermostability properties (Gase et al., infra), is used for identifying regions of the staphylokinase which can suitably be modified in their polymer attachment groups. The strategy for the identification is as described in the “Detailed Disclosure of the Invention” and the “Materials and Methods” section above. The structure (A. Rabijns, H. L. de Bondt, C. de Ranter, “Three-dimensional structure of staphylokinase a plasminogen activator with therapeutic potential” Nat. Struct. Biol. v. 4, p. 357, (1997)) is available as accession code 2SAK in the PDB (Protein Data Bank) structure depository.

### Determining Amino Acid Residues Potentially Exposed to the Surface:

The software WHAT IF (see above) was used to determine amino acid residues potentially exposed at the surface of the protein. Using the option Accessibility to “Calculate the accessible molecular surface. Output per atom” the following residues were found to have all of their side chain atoms shielded from the solvent (i.e having a zero ASA (accessible surface area), CA for Gly): L25, V27, G31, L55, A67, I87, G110, V113, L127, V131, I133. Accordingly, these are not appropriate targets for mutagenesis. Among the first 15 residues of the sequence, which are not disclosed in the X-ray structure (SSSFDKGKYKKGDDA), F4 and A15 are not hydrophilic amino acid residues or Gly and thus not expected to be exposed at the surface of the protein. Accordingly, these residues are not considered appropriate targets for mutagenesis.

Determining the Distance from the CB in Each Residue (in the Case of a Gly from the CA) to CB of all Lysine Residues (Only the Shortest Distance Reported).

| | | |
|---|---|---|
| | | Distance |
| From | To | [Å] |

| | | |
|---|---|---|
| SER 16 | LYS 121 | 14.52 |
| TYR 17 | LYS 121 | 18.01 |
| PHE 18 | LYS 121 | 13.15 |
| GLU 19 | LYS 121 | 9.95 |
| PRO 20 | LYS 121 | 9.47 |
| THR 21 | LYS 121 | 7.01 |
| GLY 22 | LYS 121 | 5.99 |
| PRO 23 | LYS 50 | 6.57 |
| TYR 24 | LYS 50 | 9.85 |
| LEU 25 | LYS 59 | 9.55 |
| MET 26 | LYS 86 | 13.10 |
| VAL 27 | LYS 59 | 11.83 |
| ASN 28 | LYS 130 | 10.25 |
| VAL 29 | LYS 130 | 7.36 |
| THR 30 | LYS 130 | 4.70 |
| GLY 31 | LYS 130 | 6.51 |
| VAL 32 | LYS 130 | 5.98 |
| ASP 33 | LYS 35 | 5.55 |
| SER 34 | LYS 35 | 5.51 |
| LYS 35 | LYS 35 | 0.00 |
| GLY 36 | LYS 35 | 4.79 |
| ASN 37 | LYS 35 | 5.55 |
| GLU 38 | LYS 130 | 8.88 |
| LEU 39 | LYS 35 | 10.61 |
| LEU 40 | LYS 130 | 10.23 |
| SER 41 | LYS 130 | 11.49 |
| PRO 42 | LYS 130 | 9.59 |
| HIS 43 | LYS 74 | 11.47 |
| TYR 44 | LYS 130 | 14.93 |
| VAL 45 | LYS 74 | 12.47 |
| GLU 46 | LYS 50 | 13.82 |
| PHE 47 | LYS 59 | 10.35 |
| PRO 48 | LYS 50 | 7.42 |
| ILE 49 | LYS 50 | 5.55 |
| LYS 50 | LYS 50 | 0.00 |
| PRO 51 | LYS 50 | 5.40 |
| GLY 52 | LYS 50 | 6.36 |
| THR 53 | LYS 50 | 5.57 |
| THR 54 | LYS 59 | 7.71 |
| LEU 55 | LYS 59 | 5.34 |
| THR 56 | LYS 59 | 4.93 |
| LYS 57 | LYS 57 | 0.00 |
| GLU 58 | LYS 57 | 5.29 |
| LYS 59 | LYS 59 | 0.00 |
| ILE 60 | LYS 59 | 5.35 |
| GLU 61 | LYS 57 | 6.49 |
| TYR 62 | LYS 59 | 5.43 |
| TYR 63 | LYS 59 | 6.64 |
| VAL 64 | LYS 59 | 10.30 |
| GLU 65 | LYS 74 | 8.58 |
| TRP 66 | LYS 74 | 9.74 |
| ALA 67 | LYS 74 | 9.80 |
| LEU 68 | LYS 74 | 5.79 |
| ASP 69 | LYS 74 | 4.50 |
| ALA 70 | LYS 74 | 7.46 |
| THR 71 | LYS 74 | 6.10 |
| ALA 72 | LYS 74 | 7.11 |
| TYR 73 | LYS 74 | 6.05 |
| LYS 74 | LYS 74 | 0.00 |
| GLU 75 | LYS 74 | 5.38 |
| ILE 76 | LYS 135 | 6.10 |
| ARG 77 | LYS 136 | 4.91 |
| VAL 78 | LYS 136 | 10.57 |
| VAL 79 | LYS 135 | 10.55 |
| GLU 80 | LYS 130 | 9.45 |
| LEU 81 | LYS 130 | 7.76 |
| ASP 82 | LYS 130 | 4.70 |
| PRO 83 | LYS 130 | 10.28 |
| SER 84 | LYS 86 | 7.39 |
| ALA 85 | LYS 86 | 5.79 |
| LYS 86 | LYS 86 | 0.00 |
| ILE 87 | LYS 86 | 5.53 |
| GLU 88 | LYS 86 | 5.70 |
| VAL 89 | LYS 102 | 5.15 |
| THR 90 | LYS 102 | 8.00 |
| TYR 91 | LYS 102 | 8.50 |
| TYR 92 | LYS 94 | 6.51 |
| ASP 93 | LYS 96 | 4.07 |
| LYS 94 | LYS 94 | 0.00 |
| ASN 95 | LYS 96 | 5.29 |
| LYS 96 | LYS 96 | 0.00 |
| LYS 97 | LYS 97 | 0.00 |
| LYS 98 | LYS 98 | 0.00 |
| GLU 99 | LYS 98 | 5.46 |
| GLU 100 | LYS 98 | 6.29 |
| THR 101 | LYS 102 | 5.84 |
| LYS 102 | LYS 102 | 0.00 |
| SER 103 | LYS 86 | 4.51 |
| PHE 104 | LYS 86 | 6.19 |
| PRO 105 | LYS 86 | 6.07 |
| ILE 106 | LYS 57 | 5.24 |
| THR 107 | LYS 109 | 5.01 |
| GLU 108 | LYS 57 | 5.27 |
| LYS 109 | LYS 109 | 0.00 |
| GLY 110 | LYS 109 | 4.45 |
| PHE 111 | LYS 109 | 7.84 |
| VAL 112 | LYS 109 | 10.28 |
| VAL 113 | LYS 50 | 6.89 |
| PRO 114 | LYS 102 | 7.45 |
| ASP 115 | LYS 50 | 9.01 |
| LEU 116 | LYS 102 | 8.35 |
| SER 117 | LYS 121 | 5.73 |
| GLU 118 | LYS 94 | 8.83 |
| HIS 119 | LYS 94 | 6.41 |
| ILE 120 | LYS 121 | 5.79 |
| LYS 121 | LYS 121 | 0.00 |
| ASN 122 | LYS 121 | 4.94 |
| PRO 123 | LYS 121 | 7.76 |
| GLY 124 | LYS 121 | 10.76 |
| PHE 125 | LYS 102 | 9.06 |
| ASN 126 | LYS 86 | 8.94 |
| LEU 127 | LYS 86 | 8.20 |
| ILE 128 | LYS 130 | 8.96 |
| THR 129 | LYS 130 | 5.29 |
| LYS 130 | LYS 130 | 0.00 |
| VAL 131 | LYS 130 | 5.83 |
| VAL 132 | LYS 130 | 7.59 |
| ILE 133 | LYS 135 | 8.43 |
| GLU 134 | LYS 135 | 5.41 |
| LYS 135 | LYS 135 | 0.00 |
| LYS 136 | LYS 136 | 0.00 |

Determining the Distance from the CB in Each Residue (in the Case of a Gly from the CA) To the Attachment Group of all Lysines, i.e. the NZ Atom of the Epsilon Amino Group of Lysine (Only the Shortest Distance Reported)

| | | |
|---|---|---|
| | | Distance |
| From | To | [Å] |

| | | |
|---|---|---|
| SER 16 | LYS 121 | 15.62 |
| TYR 17 | LYS 121 | 17.97 |
| PKE 18 | LYS 121 | 12.76 |
| GLU 19 | LYS 121 | 10.20 |
| PRO 20 | LYS 121 | 11.82 |
| THR 21 | LYS 121 | 8.23 |
| GLY 22 | LYS 121 | 9.53 |
| PRO 23 | LYS 50 | 10.64 |
| TYR 24 | LYS 59 | 14.47 |
| LEU 25 | LYS 59 | 11.07 |
| MET 26 | LYS 59 | 16.95 |
| VAL 27 | LYS 59 | 15.12 |
| ASN 28 | LYS 130 | 13.01 |
| VAL 29 | LYS 130 | 11.09 |
| THR 30 | LYS 130 | 6.31 |
| GLY 31 | LYS 130 | 8.61 |
| VAL 32 | LYS 130 | 5.93 |
| ASP 33 | LYS 35 | 8.21 |
| SER 34 | LYS 35 | 9.00 |
| LYS 35 | LYS 35 | 4.18 |
| GLY 36 | LYS 130 | 6.49 |
| ASN 37 | LYS 35 | 6.91 |
| GLU 38 | LYS 130 | 7.29 |
| LEU 39 | LYS 135 | 11.23 |
| LEU 40 | LYS 130 | 11.70 |
| SER 41 | LYS 130 | 11.70 |
| PRO 42 | LYS 130 | 10.81 |
| HIS 43 | LYS 130 | 14.22 |
| TYR 44 | LYS 130 | 17.31 |
| VAL 45 | LYS 74 | 15.86 |
| GLU 46 | LYS 59 | 16.67 |
| PHE 47 | LYS 59 | 11.25 |
| PRO 48 | LYS 50 | 12.33 |
| ILE 49 | LYS 59 | 8.75 |
| LYS 50 | LYS 50 | 5.03 |
| PRO 51 | LYS 50 | 5.94 |
| GLY 52 | LYS 50 | 4.67 |
| THR 53 | LYS 59 | 5.18 |
| THR 54 | LYS 59 | 5.80 |
| LEU 55 | LYS 59 | 6.83 |
| THR 56 | LYS 59 | 7.40 |
| LYS 57 | LYS 57 | 4.36 |
| GLU 58 | LYS 57 | 7.11 |
| LYS 59 | LYS 59 | 5.08 |
| ILE 60 | LYS 57 | 8.34 |
| GLU 61 | LYS 57 | 6.39 |
| TYR 62 | LYS 59 | 8.96 |
| TYR 63 | LYS 59 | 10.04 |
| VAL 64 | LYS 57 | 11.14 |
| GLU 65 | LYS 136 | 7.52 |
| TRP 66 | LYS 136 | 11.89 |
| ALA 67 | LYS 74 | 13.60 |
| LEU 68 | LYS 136 | 9.85 |
| ASP 69 | LYS 136 | 7.09 |
| ALA 70 | LYS 136 | 10.29 |
| THR 71 | LYS 136 | 9.72 |
| ALA 72 | LYS 136 | 9.18 |
| TYR 73 | LYS 136 | 9.57 |
| LYS 74 | LYS 136 | 4.21 |
| GLU 75 | LYS 136 | 5.59 |
| PHE 76 | LYS 136 | 7.29 |
| ARG 77 | LYS 136 | 5.58 |
| VAL 78 | LYS 136 | 8.86 |
| VAL 79 | LYS 57 | 10.13 |
| GLU 80 | LYS 57 | 7.02 |
| LEU 81 | LYS 57 | 6.64 |
| ASP 82 | LYS 130 | 7.85 |
| PRO 83 | LYS 57 | 8.48 |
| SER 84 | LYS 86 | 8.96 |
| ALA 85 | LYS 86 | 9.64 |
| LYS 86 | LYS 86 | 4.59 |
| ILE 87 | LYS 86 | 8.47 |
| GLU 88 | LYS 86 | 8.38 |
| VAL 89 | LYS 102 | 8.92 |
| THR 90 | LYS 102 | 9.78 |
| TYR 91 | LYS 102 | 9.24 |
| TYR 92 | LYS 94 | 7.41 |
| ASP 93 | LYS 96 | 5.90 |
| LYS 94 | LYS 94 | 4.00 |
| ASN 95 | LYS 97 | 7.21 |
| LYS 96 | LYS 96 | 4.93 |
| LYS 97 | LYS 97 | 4.43 |
| LYS 98 | LYS 98 | 4.52 |
| GLU 99 | LYS 98 | 8.48 |
| GLU 100 | LYS 102 | 4.79 |
| THR 101 | LYS 102 | 6.72 |
| LYS 102 | LYS 102 | 4.51 |
| SER 103 | LYS 86 | 4.25 |
| PHE 104 | LYS 86 | 6.39 |
| PRO 105 | LYS 86 | 4.68 |
| ILE 106 | LYS 57 | 7.56 |
| THR 107 | LYS 57 | 8.64 |
| GLU 108 | LYS 57 | 7.47 |
| LYS 109 | LYS 109 | 4.35 |
| GLY 110 | LYS 109 | 6.98 |
| PHE 111 | LYS 109 | 8.70 |
| VAL 112 | LYS 109 | 8.85 |
| VAL 113 | LYS 50 | 8.67 |
| PRO 114 | LYS 102 | 9.57 |
| ASP 115 | LYS 50 | 8.88 |
| LEU 116 | LYS 102 | 9.78 |
| SER 117 | LYS 121 | 9.09 |
| GLU 118 | LYS 96 | 11.61 |
| HIS 119 | LYS 96 | 10.07 |
| ILE 120 | LYS 121 | 8.77 |
| LYS 121 | LYS 121 | 4.51 |
| ASN 122 | LYS 121 | 8.09 |
| PRO 123 | LYS 121 | 11.90 |
| GLY 124 | LYS 102 | 14.43 |
| PHE 125 | LYS 86 | 13.10 |
| ASN 126 | LYS 86 | 12.72 |
| LEU 127 | LYS 86 | 12.54 |
| ILE 128 | LYS 130 | 11.56 |
| THR 129 | LYS 130 | 9.32 |
| LYS 130 | LYS 130 | 4.50 |
| VAL 131 | LYS 130 | 9.82 |
| VAL 132 | LYS 130 | 9.62 |
| ILE 133 | LYS 136 | 10.32 |
| GLU 134 | LYS 135 | 9.76 |
| LYS 135 | LYS 135 | 4.99 |
| LYS 136 | LYS 136 | 4.99 |

Determining the Shortest Distance from the Attachment Group (NZ) of Each of the Lysine Residues to the Attachment Group (NZ) of the Closest Other the Lysine Groups.

| | | |
|---|---|---|
| | | Distance |
| From | To | [Å] |

| | | |
|---|---|---|
| LYS 35 | LYS 130 | 14.54 |
| LYS 50 | LYS 59 | 10.70 |
| LYS 57 | LYS 59 | 15.39 |
| LYS 59 | LYS 50 | 10.70 |
| LYS 74 | LYS 136 | 9.64 |
| LYS 86 | LYS 102 | 11.47 |
| LYS 94 | LYS 97 | 3.66 |
| LYS 96 | LYS 102 | 6.94 |
| LYS 97 | LYS 94 | 3.66 |
| LYS 98 | LYS 96 | 8.99 |
| LYS 102 | LYS 96 | 6.94 |
| LYS 109 | LYS 59 | 13.84 |
| LYS 121 | LYS 94 | 14.05 |
| LYS 130 | LYS 35 | 14.54 |
| LYS 135 | LYS 74 | 11.98 |
| LYS 136 | LYS 74 | 9.64 |

### Determining Residues Located at the Functional Site

Based on the X-ray structure of the ternary complex of microplasmin-staphylokinase-microplasmin (Parry et. al. Nature Structural Biology, 1998, 5: 917-923) the following residues are potentially involved in staphylokinase's action. These are: E19, Y24, M26, N28, E38, S41, H43, Y44, E46, F47, P48, Y62, W66, A70, Y73, E75.

### Determining Main Epitopes of Staphylokinase

Jenné et al. The journal of Immunology, 1998, 161: 3161-3168. have determined the major B Cell epitopes of Staphylokinase in Humans. The result was 25 residue positions considered as critical for recognition of Staphylokinase (Sak) by polyclonal anti-Sak IgG's: K6, K8, S16, E19, T21, W66, D69, A72, Y73, K74, E75, F76, K94, N95, K96, K97, E99, K102, S103, K109, E118, K121, K130, K135, K136.

### Selection of Residues to be Mutagenized to a Residue Having a Polymer Attachment Group (Exemplified by Lysine)

Residues to be mutagenized to a lysine residue can be summarized as:

A) Residues potentially on the surface and not already lysine residues or N-terminal: S2, S3, D5, G7, Y9, G12, D13, D14, S16, Y17, F18, E19, P20, T21, G22, P23, Y24, M26, N28, V29, T30, V32, D33, S34, G36, N37, E38, L39, L40, S41, P42, H43, Y44, V45, E46, F47, P48, I49, P51, G52, T53, T54, T56, E58, I60, E61, Y62, Y63, V64, E65, W66, L68, D69, A70, T71, A72, Y73, E75, F76, R77, V78, V79, E80, L81, D82, P83, S84, A85, E88, V89, T90, Y91, Y92, D93, N95, E99, E100, T101, S103, F104, P105, I106, T107, E108, F111, V112, P114, D115, L116, S117, E118, H119, I120, N122, P123, G124, F125, N126, I128, T129, V132, E134.

B) Residues where the mutation is conservative: R77K

C) Residues having their CB (or in the case of a gly CA) at a distance of more than 8 Å from the CB of the nearest Lys residue: S16, Y17, F18, E19, P20, Y24, M26, N28, E38, L39, L40, S41, P42, H43, Y44, V45, E46, F47, V64, E65, W66, V78, V79, E80, P83, T90, Y91, V112, D115, L116, E118, G124, F125, N126, I128.

C) Residues having their CB (or in the case of a gly CA) at a distance of more than 10 Å from the CB of the nearest Lys residue: S16, Y17, F18, M26, N28, L39, L40, S41, H43, Y44, V45, E46, F47, V64, V78, V79, P83, V112, G124.

D) Residues having their CB (or in the case of a gly CA) at a distance of more than 10 Å from the NZ of the nearest Lys residue: S16, Y17, F18, E19, P20, P23, Y24, M26, N28, V29, L39, L40, S41, P42, H43, Y44, V45, E46, F47, P48, Y63, V64, W66, A70, V79, E118, H119, P123, G124, F125, N126, I128.

E) Residues in a known epitope region: S16, E19, T21, W66, D69, A72, Y73, E75, F76, N95, E99, S103, E118.

F) Residues which are not located at the functional site: S2, S3, D5, G7, Y9, G12, D13, D14, S16, Y17, F18, P20, T21, G22, P23, V29, T30, V32, D33, S34, G36, N37, L39, L40, P42, V45, I49, P51, G52, T53, T54, T56, E58, I60, E61, Y63, V64, E65, L68, D69, T71, A72, F76, R77, V78, V79, E80, L81, D82, P83, S84, A85, E88, V89, T90, Y91, Y92, D93, N95, E99, E100, T101, S103, F104, P105, I106, T107, E108, F111, V112, P114, D115, L116, S117, E118, H119, I120, N122, P123, G124, F125, N126, I128, T129, V132, E134.

Based on the above considerations regions including amino acid residues 16-18 and 124-128 are chosen for being subjected to localized or focused mutagenesis towards introduction of lysine residues.

### Focused Mutagenesis Towards Introduction of Lysine Residues

The below primers are used to introduce one lysine residue, at random, into each of the two regions constituting amino acid residues 16 to 18 and amino acid residues 124 to 128, respectively. The primers with the number 2 contain an Eco RI cloning site.

The primers are mixed in equimolar amounts and used in a PCR reaction. The resulting PCR product is used in a second PCR reaction with an upstream primer containing a proper cloning site in order to clone the product in a proper expression vector such as pET12a.

Primer 1a (S16K):

(SEQ ID NO 4)

5′ AAA AAG GGC GAT GAG GCG AAG TAT TTT GAA CCA ACA
GGC CCG 3′
Primer 1b (Y17K):

(SEQ ID NO 5)

5′ AAA AAG GGC GAT GAG GCG AGT AAG TTT GAA CCA ACA
GGG CCG 3′
Primer 1c (F18K):

(SEQ ID NO 6)

5′ AAA AAG GGC GAT GAG GCG AGT TAT AAG GAA GCA ACA
GGC GCG 3′
Primer 1d (wt):

(SEQ ID NO 7)

5′ AAA AAG GGC GAT GAG GGG AGT TAT TTT GAA GGA AGA
GGG GGG 3′
Primer 2a (G124K):

(SEQ ID NO 8)

5′ CGGAATTC TTA TTT CTT TTC TAT AAC AAC CTT TGT
AAT TAA GTT GAA CTT AGG GTT TTT AAT ATG C 3′
Primer 2b (F125K):

(SEQ ID NO 9)

5′ CGGAATTC TTA TTT CTT TTC TAT AAC AAC CTT TGT
AAT TAA GTT CTT TCC AGG GTT TTT AAT ATG C 3′
Primer 2c (N126K):

(SEQ ID NO 10)

5′ CGGAATTC TTA TTT CTT TTC TAT AAC AAC CTT TGT
AAT TAA CTT GAA TCC AGG GTT TTT AAT ATG C 3′
Primer 2d (I128K):

(SEQ ID NO 11)

5′ CGGAATTC TTA TTT CTT TTC TAT AAC AAC CTT TGT
CTT TAA GTT GAA TCC AGG GTT TTT AAT ATG C 3′
Primer 2e (wt):

(SEQ ID NO 12)

5′ CGGAATTC TTA TTT CTT TTC TAT AAC AAC CTT TGT
AAT TAA GTT GAA TCC AGG GTT TTT AAT ATG C 3′

Subsequently, the resulting mutated nucleotide sequences are introduced into pET12a and transformed into E. coli as described in Example 1. A small aliquot of the transformation mixture is plated on agar plates (LB medium containing ampicillin) and the rest is frozen at −80° C. The next day the transformation frequency is determined and the frozen transformation mixture is diluted so as to obtain growth in 70% of the wells when 200 mikroliters of the transformation mixture is loaded into each well of a 96-well microtiter plate. The microtiter plate is fermented until optimal expression is achieved (normally for about three days) at 30° C. Then, 20 mikroliters of the supernatant from each well is transferred to the screening plate and subjected to screening as described in Example 5.

### Example 3

### Localized Mutagenesis to Remove Amino Acid Residues Containing an Attachment Group

The criteria for the selections of suitable regions for localized mutagenesis include the following:

A) The mutation should preferably be of a conservative type.

B) Regions containing amino acid residues containing a polymer attachment group which are located close in space and/or close in sequence are target for mutagenesis. For instance, if such residues are separated by less than three amino acid residues in the primary sequence and/or having their attachment groups are separated by less than 10 Å, preferably 8 Å more preferably 5 Å the surrounding region is a target for mutagenesis.

On the basis of the above considerations and the data provided in the tables above regions including the following lysine residues are targets for mutagenesis aiming at removing and thus reducing the number of lysine residues:

K74, K94, K96, K97, K98, K102, K136 (being less than 10 Å from the attachment group of the closest other lysine residue), preferably K94, K96, K97, K102 (being less than 8 Å from the attachment group of the closest other lysine residue), and most preferably K94, K97 (being less than 5 Å from the closest other lysine residue).

The below primer 3 is designed to remove selected lysine residues from position K94, K96, K97 and K102. The primer contains an Eco47 III (or Hae II) cloning site at the 5′-end (underlined). The Ala85 codon has been changed from GCA to GCT. The primer 4 contains a Eco RI site for cloning.

The primers are mixed in equimolar amounts and used in a PCR reaction. The resulting PCR product can be cloned into a proper expression vector after digestion with Eco 47 III and Eco RI.

Primer 3 (K94X, K96X, K97X, K102X):

(SEQ ID NO 13)

5′ CCA AGC GCT AAG ATC GAA GTC ACT TAT TAT GAT 556
AAT 556 556 AAA GAA GAA ACG 556 TCT TTC CCT ATA
ACA GAA AAA 3′

Bottle 5: 70% A, 10% G, 10% C, 10% T

Bottle 6: 90% G, 10% C

Primer 4:

(SEQ ID NO 14)

5′ CGGAATTC TTA TTT CTT TTC TAT AAC AAC 3′

Subsequently, the resulting mutated nucleotide sequences are introduced into pET12a and expressed in E. coli as described in Example 1.

### Example 4

PEGylation of the diversified population of nucleotide sequences prepared as described in Example 2 and 3

Fermentation broth originating from expression in microtiter plates of the staphylokinase random mutagenesis library is transferred to a microtiter plate where each well is coated with suitable amounts of human plasmin or plasminogen and subsequently residual binding capacity blocked by BSA. Prior to addition of the fermentation broth, the plasmin or plasminogen coated microtiter wells are washed in suitable buffer, e.g. the buffer used to carry out the PEGylation step.

PEGylation is done in accordance to manufacturer's instructions. It is essential that an excess amount of activated PEG is used in order to ensure proper PEGylation of the staphylokinase. In this connection, the amount of activated PEG required reacting with the attachment groups on plasmin and BSA is to be taken into account. An alternative to BSA such as Tween 80 can be used to achieve blocking of binding capacity in the microtiter well.

PEGylation with Succinimidyl Propionate PEG is performed in accordance with methods known in the art. Monosubstituted PEG with an average molecular weight of 2000 is used (available from Shearwater, Inc., Huntsville, Ala.) and PEGylation done according to the manufacturer's instructions. When PEGylation is carried out in microtiter plates all concentrations of ingredients are used as according to the manufacturer's instructions, only volumes are scaled down. For instance, during conjugation in a 96-well microtiter plate a final volume of approx. 200 mikroliters are used per well.

### Example 5

### Screening and Selection of Improved PEGylated Staphylokinase Variants

### Efficacy Assay:

The efficacy of PEGylated staphylokinase variants resulting from Example 4 is analysed by the chromogenic assay described above in the Materials and Methods section. The principle is that staphylokinase in complex with plasmin activates plasminogen to plasmin. Plasmin liberates a chromophore from commercially available chromogenic substrates for plasmin e.g. S-2251 from Chromogenix. Differences in the kinetic behavior in this assay reflects the variants' and the PEGylated variants' plasminogen-activating efficacy. The efficacy of the PEGylated variants is assayed in microtiterplates according to the method described in the Materials and Methods section herein.

### Immunological Assay:

Immunological assays are conducted using the method described in the Materials and Methods section.

### Example 6

### Purification and Characterization of PEGylated Staphylokinase Variants

A DNA sequence encoding the polypeptide part of the staphylokinase-PE conjugate selected as described in Example 5 is isolated and used for recombinant production of larger amounts of said polypeptide part using the expression system described in Example 1. Subsequently, the resulting polypeptides are purified (as described below) and subjected to PEGylation as described in Example 4.

To ease purification of the relatively large number of variants the commercially available system called TagZyme is used. Briefly described it requires that the protein is expressed with a His15-tag that facilitates binding to an Immobilised metal-ion Affinity Chromatography (IMAC) matrix (e.g. a Zn-chelate matrix). Following the initial purification of His15-tagged staphylokinase variants employing IMAC, the His15-tag is cleaved off with a His15-tagged diamino peptidase. Subsequently, the His15-tagged diamino peptidase (as well as other contaminants) is removed from the staphylokinase variants using substractive IMAC. Alternatively, standard purification schemes known from the literature will be employed.

Before PEGylation is carried out on the purified staphylokinase variants the following characterization is done.

SDS-PAGE (Coomassie BB stained) for purity,
LAL-test for endotoxins,
Mass spectrometry and amino acid sequencing for identity and to confirm that the expected changes are present, and
Amino acid analysis for concentration determination.

Following the PEGylation of purified staphylokinase variant the surplus of reagents is removed through a final gelfiltration.

The purified PEGylated staphylokinase variants are analysed and characterized by SDS-PAGE for size heterogeneity, IEF for charge heterogeneity, Analysis of degree of PEGylation, e.g. by assaying the conjugate with trinitrobenzene sulfonic acid (TNBS) to determine the number of free amino groups, analytical size exclusion HPLC with light scattering detection, analytical an-ion, cat-ion, or hydrophobic interaction chromatography, amino acid analysis for concentration determination, peptide mapping and mass spectrometry and amino acid sequencing of resulting peptides.

### Example 7

### Introduction of Glycosylation Sites in Staphylokinase

In Staphylococcus aureus staphylokinase having the amino acid sequence shown in SEQ ID NO 2 the following mutations Xxx-Asn placed at positions potentially exposed to the surface at sequence positions located two residues prior to a Ser or a Thr and not at the N-terminal position and not containing a Pro at the “middle” position introduce a potential N-glycosylation site: D14N, L39N, P51N, G52N, T54N, D69N, E88N, E99N, T101N, P105N and/or D115N. Most preferably, the mutations to be used for introducing an N-glycosylation site are D14N, D69N and/or D115N.

Similarly in the Staphylococcus aureus staphylokinase the following mutations Xxx-Ser or Xxx-Thr placed at amino acid residues located two residues after a potential surface exposed Asn residue and not just after a Pro residue introduce a potential N-glycosylation site: L39S, K97S, I128S, L39T, K97T and/or I129T.

The mutations are introduced by site-directed or random mutagenesis by use of conventional methods known in the art. For expression of the protein of interest in S. cerevisiae a plasmid shuttle vector based on the pYES vectors (InVitrogen Inc.) can be used. For instance the published expression vector pJSO37 (Okkels, Ann. New York Acad. Sci. 782, 202-207, 1996) is used for expression of the staphylokinase by cloning the gene encoding the mature part of the staphylokinase in frame and just downstream of the signal peptide of the lipase gene (present in pJSO37). The staphylokinase variants will then be glycosylated in the S. cerevisiae cell and secreted.

### Example 8

### Random Alteration of Glycosylation Pattern

In many therapeutic applications it is desirable to alter the immunogenicity or functional in vivo or serum half-life of an administered polypeptide or protein therapeutic agent. In many cases, it is preferable to administer a protein with reduced immunogenic potential to prevent or reduce an immune response against the agent which results in neutralization or elimination of the agent, or in immune mediated side effects, including cell or tissue damage and anaphylaxis. Alterations in the glycosylation pattern of a polypeptide influences immunogenicity in at least two important ways. Firstly, many antibodies recognize epitopes present on the glycosylated form of proteins. Secondly, glycosylation can influence processing of an antigenic protein, and or mask certain antigenic epitopes of a polypeptide.

To produce a target protein therapeutic agent with reduced immunogenicity, a library of target protein variants, or subportions thereof, is produced by any combination of the mutagenesis methods described herein. For example, random mutagenesis is favorably employed in cases where little is known regarding the presence, and location of antigenic regions of the protein. Focused mutagenesis, employing spiking mixtures enriched for nucleotide sequences likely to encode N- or O-glycosylation sites, e.g., that encode asparagine, serine, or threonine residues, is also desirable in this context. In cases where epitope mapping has indicated particular regions of the protein contributing to a 1° or 3° structure comprising an epitope, localized mutagenesis is favorably utilized. The library is then screened to assess immunogenicity, half-life, or other desirable properties, of the protein variants. For example, the ability of variants to elicit a lymphoproliferative response in cells specific for the target protein is assayed in vitro by measuring ³H-thymidine uptake. Alternatively, antibody binding can be quantitated. Confirmation of a reduction in immunogenicity can be achieved by immunizing an experimental organism, e.g., a mouse, and assessing the resulting immune response by techniques well established in the art (see, e.g., Current Protocols in Immunology (1991) Coligan et al. (eds) John Wiley and Sons, New York).

In other cases, the library is screened for variants that exhibit an increased ability to elicit an immune response. Such protein variants can be valuable reagents in the production of specific antibodies for experimental and therapeutic purposes. Alternatively, target proteins with altered immunogenicity that have a reduced ability to elicit one aspect of an immune response, e.g., IgE secretion, while maintaining the capacity to evoke another aspect of a specific immune response, e.g., IgG secretion, can be identified among the variants of the library. Such proteins are useful, e.g., in producing desensitization to a specific allergen corresponding to the target protein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications and patent documents cited in this application are incorporated by reference in their entirety for all purposes to the same extent as if each individual publication or patent document were individually so denoted.

### SEQUENCE LISTING

### SEQ ID NO 1

| SEQ ID NO 1 | |
|---|---|
| ID SPSAK1 standard; DNA; PRO; 1377 BP. | |
| XX | |
| AC X00127; | |
| XX | |
| SV X00127.1 | |
| XX | |
| NI g47425 | |
| XX | |
| DT 20-FEB-1985 (Rel. 05, Created) | |
| DT 30-MAR-1995 (Rel. 43, Last updated, Version 2) | |
| XX | |
| DE Staphylococcus aureus S-phi-C gene for staphylokinase | |
| XX | |
| KW kinase; plasminogen activator; signal peptide; staphylokinase. | |
| XX | |
| OS Staphylococcus aureus | |
| OC Bacteria; Firmicutes; Bacillus/Clostridium group; Bacillaceae; | |
| OC Staphylococcus. | |
| XX | |
| RN [1] | |
| RP 1-1377 | |
| RX MEDLINE; 84069795. | |
| RA Sako T., Tsuchida N.; | |
| RT “Nucleotide sequence of the staphylokinase gene from Staphylococcus | |
| RT aureus”; | |
| RL Nucleic Acids Res. 11:7679-7693(1983). | |
| XX | |
| DR SWISS-PROT; P00802; SAK_STAAU. | |
| XX | |
| FH Key Location/Qualifiers | |
| FH | |
| FT source 1..1377 | |
| FT /organisim = “Staphylococcus aureus” | |
| FT /db_xref = “taxon:1280” | |
| FT /strain = “Phage S-phi-C” | |
| FT promoter 193..198 | |
| FT /note = “poss. promoter region” | |
| FT promoter 217..222 | |
| FT /note = “poss. promoter region” | |
| FT RBS 301..305 | |
| FT /note = “ribosome binding site” | |
| FT sig_peptide 313..393 | |
| FT /note = “put. signal peptide” | |
| FT CDS 313..804 | |
| FT /db_xref = “PID:g758303” | |
| FT /db_xref = “SWISS-PROT:P00802” | |
| FT /transl_table = 11 | |
| FT /product = “staphylokinase” | |
| FT /protein_id = “CAA24957.1” | |
| FT | |
| /translation = “MLKRSLLFLTVLLLLFSFSSITNEVSASSSFDKGKYKKGDDASYF | |
| FT | |
| EPTGPYLMVNVTGVDGKGNELLSPHYVEFPIKPGTTLTKEKIEYYVEWALDATAYKEF | |
| R | |
| FT | |
| VVELDPSAKIEVTYYDKNKKKEETKSFPITEKGFVVPDLSEHIKNPGFNLITKVVIEKK | |
| FT ″ | |
| FT mat_peptide 394..801 | |
| FT /note = “staphylokinase” | |
| XX | |
| SQ Sequence 1377 BP; 452 A; 184 C; 255 G; 486 T; 0 other; | |
| GTATACGCGC TGGAACATTA ATATATGTGT TTGAAATTAT AGATGGTTGG TGTCGCATTT | 60 |
| ATTGGAACAA TCATAATGAG TGGATATGGC ATGAGAGATT GATTGTGAAA GAAGTGTTTT | 120 |
| AATTCTTAGG TTAAAATGTT AAATATTTGT TAATTATTTT TGAATGTAAG TTTAGTTTCT | 180 |
| TTTAATATTT TATTGATTTT TAATATTTTC TCAATATAAA ATGAAGTTGT TGATATTTAT | 240 |
| CATCTTAAAT AAGGGTGTTA GCTATAAAAA GAGATAAATA AAAACAAATA TATTATATTT | 300 |
| GGAGGAAGCG CCATGCTCAA AAGAAGTTTA TTATTTTTAA CTGTTTTATT GTTATTATTC | 360 |
| TCATTTTCTT CAATTACTAA TGAGGTAAGT GCATCAAGTT CATTCGACAA AGGAAAATAT | 420 |
| AAAAAGGGCG ATGACGCGAG TTATTTTGAA CCAACAGGCC CGTATTTGAT GGTAAATGTG | 480 |
| ACTGGAGTTG ATGGTAAAGG AAATGAATTG CTATCCCCTC ATTATGTCGA GTTTCCTATT | 540 |
| AAACCTGGGA CTACACTTAC AAAAGAAAAA ATTGAATACT ATGTCGAATG GGCATTAGAT | 600 |
| GCGACAGCAT ATAAAGAGTT TAGAGTAGTT GAATTAGATC CAAGCGCAAA GATCGAAGTC | 660 |
| ACTTATTATG ATAAGAATAA GAAAAAAGAA GAAACGAAGT CTTTCCCTAT AACAGAAAAA | 720 |
| GGTTTTGTTG TCCCAGATTT ATCAGAGCAT ATTAAAAACC CTGGATTCAA CTTAATTACA | 780 |
| AAGGTTGTTA TAGAAAAGAA ATAAAACAAA ATAGTTGTTT ATTATAGAAA GTAATGTCTT | 840 |
| GATTGAATAT GTGTAGTGAA ATTATCTTTC ATCAAATTCT CATTCATGCA CGAATGGTTC | 900 |
| TGCCCCACCT AATCAGATAT TAGGTGACTT ATGGGGAGAA ATCAGTTAGA ATGACATAGT | 960 |
| CATGTCTATT TAAGCAGGTG CGTTACACAC CTGATGTCTA TTTACATTTA AAGATAAAAT | 1020 |
| GTGCTATTAT TTTACTAGAA CTTTTTAACA TTTCTCTCAA GATTTAAATG TAGATAACAG | 1080 |
| GCAGGTACTA CGGTACTTGC CTGTTTTTTT ATGTTATAGC TAGCCTTCGG GCAGTTTTTG | 1140 |
| TTATGATGCG TTACACACGC ATCAACTATT CACACCTATC TTTGTTCACC TAAGCATGTC | 1200 |
| ACTGGGTGTT TTTTTCTTAC GATAGAGAGC ATAGTTTTCA TACTACTCCC CGTAGTATAT | 1260 |
| ATGACTTTAG CATTCCCGTA TAACAGTTTA CGGGGTGCTT TTTATGTTAT ACTTACTTTT | 1320 |
| ATATAGTAGG AGTGGACTAT ATAGCTGGTC AGAGGCTGTA TATCTGACTG TTGGTCC | 1377 |
| // | |
| (Mature S. aureus staphylokinase) | |

| | SEQ ID NO 2 |
|---|---|
| SSSFDKGKYK KGDDASYFEP TGPYLMVNVT GVDGKGNELL SPHYVEFPIK | |
| PGTTLTKEKI EYYVEWALDA TAYKEFRVVE LDPSAKIEVT YYDKNKKKEE | |
| TKSFPITEKG FVVPDLSEHI KNPGFNLITK VVIEKK | |
| (G345 S. aureus staphylokinase mutant) | |
| SEQ ID NO 3 | |
| SSSFDKGKYK KGDDASYFEP TGPYLMVNVT GVDSKGNELL SPHYVEFPIK | |
| PGTTLTKEKI EYYVEWALDA TAYKEFRVVE LDPSAKIEVT YYDKNKKKEE | |
| TKSFPITEKG FVVPDLSEHI KNPGFNLITK VVIEKK | |

1-24. (canceled)

25. A method for producing a variant binding protein with reduced immunogenicity or increased in vivo half-life comprising:
a) selecting regions of a nucleotide sequence encoding a binding protein of interest;
b) mutating the selected regions of the nucleotide sequence by site-specific mutagenesis thereby producing a diversified population of nucleotide sequences encoding variant binding proteins;
c) expressing polypeptides encoded by the diversified population of nuleotide sequences to produce a population of variant binding proteins;
d) blocking the active site of the population of binding proteins by binding to a ligand;
e) covalently conjugating a carbohydrate moiety to the blocked variant binding proteins of step (d) in vitro under conditions whereby the carbohydrate moieties are not conjugated to the blocked active site region of the variant binding proteins; and
f) screening for variant binding proteins with reduced immunogenicity or a longer half-life in vivo compared to the binding protein of interest.

26. The method of claim 25 wherein the binding protein is an antibody.

27. The method of claim 25 wherein the binding protein is a receptor protein.

28. The method of claim 25 wherein the binding protein is an enzyme.

29. The method of claim 25 wherein the regions of nucleotide sequence are selected by analyzing the tertiary structure of the binding protein.
